(19) United States
(12) Patent Application Publication
(10) Pub. No.: US 2004/0214186 A1
(43) Pub. Date: Oct. 28, 2004

(54) Method for identifying constitutively active mutants of mitogen activated protein kinase (mapk) and uses thereof

(76) Inventors: David Engelberg, Jerusalem (IL); Michal Bell, Vitkin (IL); Alexander Levitzki, Jerusalem (IL)

Correspondence Address: Cooper & Dunham, 1185 Avenue of the Americas, New York, NY, 10036, US

(21) Appl. No.: 10/474,239

(22) PCT Filed: Apr. 4, 2002
(86) PCT No.: PCT/PL02/00281

(30) Foreign Application Priority Data
Apr. 5, 2001 (IL) 142481

(51) Int. Cl. C12Q 1/68; C12N 1/18; C12N 15/74

(52) U.S. Cl. 435/6; 435/483; 435/254.2

(57) ABSTRACT

The present invention relates to a method of screening for constitutively active mutants of a desired eukaryotic MAPK pathway member of a MAPK pathway member, comprising the steps of (a) providing a mutant yeast strain devoid of an upstream kinase; (b) providing a DNA library of different mutants of a mutagenized gene coding for the desired MAPK pathway member; (c) introducing said library into said yeast strain under conditions suitable for activation of the yeast MAPK pathway; (d) detecting an end-point indication for activation of the yeast pathway; and (e) optionally isolating said constitutively activated mutant from selected rescued clones. The invention further relates to isolated constitutively active mutants of the MAPK pathway member, and their various uses particularly, in a method of screening for substances which are inhibitors of a MAPK pathway, and in drug design.

### FIELD OF THE INVENTION

The invention relates to a method of screening for specific, constitutively active mutants of MAPKs (mitogen activated protein kinases), to the isolated active MAPK mutants and to different uses thereof, particularly in screening for inhibitors of MAPK signal transduction pathways.

### BACKGROUND OF THE INVENTION

Throughout this application various publications are referred to in square brackets. A list of these publications appears at the end of the description, immediately preceding the claims. All of these publications and references included therein are fully incorporated herein.

MAPK is a generic term for a large family of enzymes, which function in a variety of signal transduction pathways. Mammalian MAPKs are divided to at least three subfamilies (ERKs, p38s and JNKs) based on degree of homology, biological activities and phosphorylation motif [Marshall (1994); Robinson and Cobb (1997); Gustin et al. (1998); Ip and Davis (1998); Widmann et al. (1999); Cobb and Goldsmith (2000)]. Although highly homologous in structure and in pattern of activation, each MAPK is activated in response to a specific battery of signals and in turn phosphorylates a particular array of substrates. As a result, each MAPK imposes specific effects on the cell. For example, in many cell lines (e.g., fibroblasts), the ERK MAP kinases are activated when cells are exposed to growth factors and their activation is important for enhancement of cell proliferation [Cowley et al. (1994); Mansour et al. (1994)). In other cells however (neuronal and myogenic cell lines), activation of ERKs is associated with growth arrest and differentiation [Cowley et al. (1994); Gredinger et al. (1998)].

In contrast to the ERK enzymes, the activity of p38 and JNK MAP kinases is only slightly induced by growth factors. These enzymes are strongly activated in response to stress signals such as heat shock, osmotic shock, UV radiation, cytokines and metabolic inhibitors. JNK and p38 MAPKs seem to be responsible mainly for protective responses, stress dependent apoptosis and inflammation lip and Davis (1998); Ono and Han (2000)). In some cell types, however, p38 and JNK may play a role in differentiation and development [Minden and Karin (1997); Ip and Davis (1998); Ono and Han (2000)]. Studies with knockout mice and knockout cell lines revealed essential roles for MAPKs in various aspects of embryonal development [Dong et al. (1998); Yang et al. (1998); Pages et al. (1999); Sabapathy et al. (1999); Tournier et al. (2000); Tamura et al. (2000)]. MAPKs are also associated with various pathological situations including inflammation and cancer [Pearson et al (2001); Kyrialis and Avruch (2001)].

Although many aspects of MAPK biology have been revealed, the exact role of each MAPK in a given biological system is not fully understood and is difficult to study. The main reason for this difficulty is scientist's inability to activate a given MAPK in vivo and to follow the biochemical and physiological consequences. Currently, a MAPK is experimentally activated in vivo using extracellular stimuli, or through expression of an active form of a component which functions upstream to that MAPK [Cowley et al. (1994); Mansour et al. (1994)]. Each of these treatments activates more than one MAPK and evokes many cellular responses. Activation of a given MAPK per se could be theoretically obtained by expressing a constitutively active form of this kinase. However, the catalytic activity of MAPKs is tightly regulated and strictly dependent on upstream activation [Robbins et al. (1993]; Raingeaud et al. (1995); Cobb and Goldsmith (2000)]. Although the mechanisms of MAPK activation have been revealed [Canagarajah et al. (1997); Khokhlatchev et al. (1998); Cobb and Goldsmith (2000)], this knowledge could not be applied for the production of constitutively active forms of MAPKs.

Following exposure of cells to an extracellular ligand, the activity of the relevant MAPK increases by about 1000 fold [Robbins et al. (1993)]. This activation is mediated through a complex signal transduction net that culminates in phosphorylation of the MAPK by a MAPK kinase (MAPKK). MAPKKs are dual specificity kinases, which phosphorylate MAPKs in particular Thr and Tyr residues. This dual phosphorylation is the basis for the dramatic increase in MAPK activity. MAPKs mutated in either the Thr or the Tyr phosphoacceptors cannot be activated [Robbins et al. (1993); Schuller et al. (1994)]. The unusual mode of MAPK activation (through dual phosphorylation) underlies the difficulties in producing active forms of these enzymes, because a PO₄-Thr-PO₄-Tyr structure is difficult or impossible to mimic by mutagenesis [Robbins et al. (1993)]. Comparison of the crystal structure of ERK2 with that of dually phosphorylated ERK2 [Canagarajah et al. (1997)] shows that phosphorylation of the activation loop induces conformational changes in both the activation loop itself and in another domain at the C-terminal extension (Pro309-Arg358) known as L16. These conformational changes induce tight interactions between the phosphorylation lip and the L16 domain. Among other effects, these interactions create an interface for homodimerization of the kinase molecules. Dimerization is stabilized by hydrophobic contacts involving mainly leucine residues located at L16 of the two monomers and by an ion pair involving Phe329 and Glu343 (located in L16) of one monomer and H176 of the other monomer [Khokhlatchev et al. (1998)].

Although the crystal structures of ERK2 and phospho-ERK2 revealed the conformational changes, which activate the enzyme, there is no any suggested strategy for producing a constitutively active MAPK by mutagenesis.

The present invention describes herein a novel genetic screening system, for the production and isolation of activated mutants of MAPK.

The inventors have devised a novel protocol for isolation of active forms of MAPKs using a genetic screening method in yeast. The yeast Saccharomyces cerevisiae possesses five different MAPKs, which are highly homologous to their mammalian counterparts [Gustin et al. (1998); Madhani and Fink (1998)]. The yeast MAPKs Fus3, Kss1 and Mpk1 are close to the ERK subfamily. The yeast Hog1 MAPK has a QM*TG*YVSTR phosphorylation motif (almost identical to that of p38) and is functionally replaced by either JNKs [Galcheva-Gargova et al. (1994)] or p38s [Han et al. (1994)]. Hog1 is phosphorylated and activated by the MAPKK Pbs2 [Brewster et al. (1993)], a functional homolog of JNKK1/MKK4 [Lin et al. (1995)]. The Pbs2/Hog1 MAPK cascade is essential for survival of yeast cells under high osmotic conditions. Yeast cells lacking either PBS2 or HOG1 cannot grow on media supplemented with high concentrations of sugar or salt [Brewster et al. (1993)]. The inventors have reasoned that effort to produce active forms of MAPKs may take advantage of the Pbs2/Hog1 pathway, and therefore, it should be possible to screen a library of randomly mutated HOG1 genes in cells lacking Pbs2 activity. The premise is that a HOG1 clone that allows pbs2Δ cells to grow on salt should encode a constitutively active, MAPKK independent, Hog1 mutants as described by FIG. 1.

Previous efforts to obtain active forms of MAPKs were only partially successful. Several gain-of-function mutations that were identified in the S. cerevisiae FUS3 [Brill et al. (1994); Hall et al. (1996)] and in the Rolled MAPK of Drosophila melanogaster [Brunner et al. (1994); Bott et al. (1994)] did not render the kinases constitutively active. In yet another study, these gain of function mutations (of FUS3 and Rolled) were introduced into equivalent locations in a corresponding MAPK, the mammalian ERK2. Each mutation alone did not give rise to a significant increase in the catalytic activity of ERK2, however, combination of several of those mutations on the same ERK2 molecule did increase RIRK2 activity. Nevertheless, such increase in activation was far below the activity of activated (dually phosphorylated) ERK2 [Emrick et al. (2001)]. It should be appreciated that the gain of function mutations described in the above studies are located at different sites than activating mutations identified by the method of the present invention. The use of MAPKK-MAPK hybrids [Robinson et al. (1998); Zheng et al. (1999)] seems more useful. Yet, as in the in vivo situation, MAPKKs and MAPKs are not co-localized in the cell and are differently controlled, therefore, the use of the chimeric proteins might be problematic for physiological studies. Thus, in spite of efforts through various approaches, constitutively active, MAPKK independent MAPKs were not available.

The approach taken in this invention is most stringent as it screens for MAPKs, which are active in the complete absence of their relevant MAPKK. Moreover, the design of the screening of the present invention, forces only minor changes in the MAPK (point mutations).

The invention further discloses herein the isolation of 9 different point mutations in the Hog1 kinase by the novel method of the invention.

Each mutation is sufficient to render Hog1 catalytically and biologically active independent of upstream regulation. Remarkably, insertion of equivalent mutations to the human counterpart of Hog1, p38α, rendered this enzyme constitutive catalytically active.

Therefore, objects of the present invention are to develop a screening method for activated MAPK mutants, and to demonstrate the isolation of such mutants and the uses thereof, particularly in screening for inhibitors for MAPK signal transduction pathways.

### SUMMARY OF THE INVENTION

In a first aspect, the invention relates to a method of screening for constitutively activated mutants of a desired eukaryotic MAPK pathway member of a MAPK pathway, comprising the steps of (a) providing a mutant yeast strain devoid of an upstream kinase; this upstream kinase activates a MAPK pathway member, in a non-mutant strain of said yeast that is equivalent or corresponding to said desired eukaryotic MAPK pathway member; (b) providing a DNA library of different mutants of a gene coding for said desired MAPK pathway member; (c) introducing said library into said yeast strain under conditions suitable for activation of the yeast MAPK pathway; (d) detecting an end-point indication for activation of the yeast pathway. This activation resulting from rescuing said pathway by a constitutively activated mutant of the MAPK pathway member; and (e) optionally isolating said constitutively activated mutant from selected rescued clones.

The activation of the constitutively activated mutant detected and optionally isolated by the method of the invention is independent of the presence of an kinase upstream thereto. This upstream kinase is capable of phosphorylating and activating said MAPK pathway member.

The yeast employed in the method of the invention are preferably Saccharomyces cerevisiae and Schizosaccharomyces pombe.

In one preferred embodiment, the end-point indication may be any one of the ability of the cells to proliferate, to survive under high osmotic conditions, to survive under hypoosmotic conditions, their ability to mate, to invade agar, to form pseudohyphae, to sporulate and any combinations thereof.

The eukaryotic MAPK pathway member may be selected from the group consisting of mammalian cells, plant cells, avian cells, insect cells, fungi cells and yeast cells MAPK pathway member. More preferably, the eukaryotic MAPK pathway member may be any one of MAPK's and MAPKK's.

In a preferred embodiment, the method of the invention is used to detect and optionally isolate MAPKs, which may be selected from the group consisting of plant cells, insect cells, avian cells or mammalian ERK1, ERK2, ERK 5, ERK 7, JNK, p38 subfamilies, and the fungi and yeast Pus3, Kss1, Mpk1 and Hog1.

In a particular embodiment, the invention relates to a method of screening for a constitutively activated mutant of MAPK selected from plant, insect, avian, or mammalian ERK subfamily, or the fungi and the yeast Fus3, Kss1 and Mpk1, which method comprises the steps of (a) providing the mutant yeast strain ste7Δ, devoid of the MAPKK, Ste7, which is the upstream kinase of said MAPK; (b) providing a DNA library of different mutants of mutagenized gene coding for one of said yeast, fungi, plant, avian, insect or mammalian MAPK's; (c) introducing said library of (b) into said ste7Δdeficient yeast strain of (a) under conditions suitable for activation of said yeast MAPK pathway; (d) detecting an end-point indication for activation of the yeast MAPK pathway, which activation results from rescuing said pathway by a constitutively activated mutant of said MAPK; and optionally (e) isolating said constitutively activated MAPK mutant from selected rescued clones.

In this embodiment the said yeast MAPK pathway may be the Ste7/Fus3 and the end-point indication is the ability of rescued cells to mate, or the Mkk/Mpk1 pathway were the end-point indication is the ability to rescue cells from hypoosmotic conditions. Alternatively, the yeast MAPK pathway may be the Ste7/Kss1 pathway where the end-point indication is the ability of rescued cells to invade agar and/or to form pseudohyphae.

Another embodiment relates to a method of screening for a constitutively activated mutant of MAPK of plant, insect, avian or mammalian JNK or p38 subfamilies, and the fungi and yeast Hog1, which method comprises the steps of (a) providing the mutant yeast strain pbs2Δ which is devoid of the upstream MAPKK, Pbs2; (b) providing a DNA library of different mutants of a mutagenized gene which codes for one of said yeast, plant, avian, insect or mammalian MAPK's; (c) introducing said library into said pbs2Δ yeast strain under conditions suitable for activation of said yeast MAPK pathway; (d) detecting an end-point indication for activation of the yeast MAPK pathway, which activation results from rescuing of said pathway by a constitutively activated mutant of said MAPK; and optionally (e) isolating said constitutively activated MAPK mutant from said selected rescued clones.

In this particular embodiment, the yeast MAPK pathway may be the Pbs2/Hog1 and the end-point indication is the ability of rescued cells to survive under high osmotic conditions.

In yet another specific embodiment, the invention relates to a method of screening for a constitutively activated mutant of yeast Hog1, which method comprises the steps of (a) providing the mutant yeast strain pbs2Δ devoid of the upstream MAPKK, Pbs2, which Pbs2 is a luinase upstream to Hog1 and is capable of phosphorylating Hog1; (b) providing a DNA library of mutants of mutagenized HOG1 gene coding for the MAPK Hog1; (c) introducing said library of mutants into said pbs2Δ yeast strain under conditions suitable for activation of said yeast Pbs2/Hog1 pathway; (d) detecting an end-point indication for activation of the yeast Pbs2/Hog1 pathway, which activation results from rescuing said pathway by a constitutively activated Hog1 mutant; and optionally (e) isolating said constitutively activated Hog1 mutant from selected rescued clones. The constitutively activated Hog1 mutant is preferably capable of activating the authentic pathway downstream to Hog1 independently of Pbs2 and endogenous Hog1.

The mutants to be detected and optionally isolated by the method of the invention may have at least one mutation selected from the group consisting of point mutations, missense, nonsense, insertions, deletions or rearrangement, preferably at least one mutation in the conserved L16 domain of the protein, particularly between residues 314 to 332 of said L16 domain. These residues are located within the Hog1 amino acid sequence, as substantially denoted by SEQ ID No. 1. However, it should be appreciated that activating mutations within an L16 domain of any equivalent MAPK, may also be detected and isolated by the method of the invention, and therefore are also contemplated to be within the scope of the present invention.

The mutants detected by the method of the invention may have at least one point mutation located within the L16 domain of the Hog1 molecule. Such mutations may be located at positions A314, F318, W320, F322, W332 and any combinations thereof, of the Hog1 amino acid sequence. Preferred mutations obtained using the method of the invention are selected from the group consisting of A314T, F318L, F318S, W320R, F322L, W332R and any combinations thereof. Alternatively, activating mutations may be located out of the L16 region, for example at position N391 or within the N terminal portion of the Hogs molecule at position Y68 or D170, and any combinations thereof. Preferred mutation may be for example any of N391D Y68H, D170A, and any combinations thereof. The particular numerical locations of the different mutations relate to the amino acid sequence of Hog1, substantially as denoted by SEQ ID No. 1.

It is to be understood that activating mutations located in corresponding positions within any equivalent MAPK, may be detected by the method of the invention and therefore are also within the scope of the invention.

In another embodiment, the MAPK pathway member to be detected and optionally isolated by the method of the invention is a MAPKK.

Thus, the invention also relates to a method of screening for a MAPKK selected from the group consisting of plant, avian, insect or mammalian MEK-family, JNKK family and MKK family the fungi and yeast Ste7, Mkk1, Mkk2 and Byr1, which method comprises the steps of (a) providing a mutant yeast strain MAPKKKΔ devoid of the upstream MAPKKK; (b) providing a DNA library of mutants of a mutagenized gene coding for one of said yeast, plant, avian, insect or mammalian MAPKK; (c) introducing said library into said MAPKKKΔ yeast strain under conditions suitable for activation of said yeast MAPK pathway; (d) detecting an end-point indication for activation of the yeast MAPK pathway, which activation results from rescuing said pathway by a constitutively activated mutant of the MAPKK; and optionally (e) isolating said constitutively activated MAPKK mutant from selected rescued clones.

In a second aspect, the invention relates to a constitutively activated mutant of a MAPK pathway member, capable of activating said MAPK pathway independently of the upstream kinase.

The mutants of the invention have at least one mutation selected from the group consisting of point mutation, missense nonsense, insertion, deletion or rearrangement, and are preferably mutants of MAPK and MAPKK, particularly eukaryotic MAPKs and MAPKKs selected from mammalian cells, plant cells, avian cells, insect cells, fungi cells and yeast cells MAPK pathway members.

In a preferred embodiment, the MAPK mutants of the invention may particularly be mammalian ERK1, ERK2, ERK5, ERK7, JNK or p38 subfamilies, the fungi and yeast Fus3, Kss1, Mpk1 and Hog1.

A particular MAPK mutant may be according to a specific embodiment, a Hog1 mutant. Such particular Hog1 mutant is capable of activating the authentic pathway downstream to Hog1 independently of Pbs2 and endogenous Hog1.

In yet another embodiment, the activated MAPK mutant of the invention may have at least one mutation occurring in the conserved L16 domain of the Hog1 protein, particularly between residues 314 to 332 of said L16 domain as located in the amino acid sequence Hog1 as denoted by SEQ ID No. 1. In case the mutant of the invention is a MAPK equivalent to Hog1, such as for example ERK, JNK and p38 family members, a preferred mutant may carry at least one point mutation in their respective L16 domain of any of said MAPKs.

These activated MAPK mutants may have at least one point mutation in Hog1 or in a respective mutation in any MAPK, which mutation may be located at position A314, F318, W320, F322, W332 and any combinations thereof.

Preferably, the Hog1 mutant of the invention may carry at least one point mutation selected from A314T, F318L, F318S, W320R, F322L, W332R and any combination thereof.

In another specific embodiment, where the active mutant maybe any respective MAPK, the said respective mutation in any MAPK may be located at position A342 or F346 of the human ERK1, A325 or F329 of the human ERK2, A323 or F327 of the rat ERK2, F366 or F362 of the human ERK5, W352 of the human JNK1 and A320, F327 or W337 of the human or rat p38 and any combination thereof. Preferred mutants according to this embodiment may be any one of A342T, F346L and F346S of the human ERK1, A325T, F329L and F329S of the human ERK2, A323T, F327S and F327L of the rat ERK2, F366L, F362L and F362S of the human ERK5, W352R of the human JNK1 and A320T, F327L, F327S, W337R of the human or rat p38 and any combination thereof, particularly A320T, W337R, F327S and F327L that are point mutations in the human or the rat p38. Most preferred mutant according to this embodiment is a p38 mutant, that carry at least one point mutation selected from A320T, W337R, F327S and F327L. The exact location refers to the human or the rat p38 amino acid sequence as denoted by the GeneBank accession no. P45983 and AF346293, respectively. Both are fully incorporated herein by reference.

In yet another embodiment, the activated mutants of the invention may carry at least one point mutation in Hog1 or a respective mutation in any MAPK, which is out of the L16 domain. Such mutation may be located at any position of Y68, D170 and N391, or in corresponding location in any other equivalent MAPK. Preferred mutants of the invention may carry at least one point mutation in Hog1 or a respective mutation in any MAPK, selected from the group consisting of Y68H, D170A and N39 ID, or in corresponding location in any other equivalent MAPK. More particularly, where the mutant is Hog1, this point mutation may be selected from the group consisting of Y68H, D170A, N391 D and any combinations thereof.

In the activated MAPK mutants of the invention, the said respective mutation in any of said MAPK may be located at position D192 of the human ERK1, D175 of the human ERK2, D173 of the rat ERK2, Y71 of the human JNK1, or Y68 and D176 of the human and the rat p38. Preferred mutants may be selected from the group consisting of D192A of the human ERK1, D175A of the human ERK2, D173A of the rat ERK2, Y71H of the human JNK1, Y68H and D176A of the human and the rat p38.

The invention also relates to an activated MAPK or MAPKK obtained by the method of the invention.

In a further aspect, the invention relates to a method of producing a desired constitutively activated mutant of a eukaryotic MAPK pathway member, which method comprises the steps of (a) providing an activated MAPK pathway member of the invention; (b) subjecting the activated mutant provided in step (a) to nucleic acid sequence analysis; (c) aligning the sequence obtained in step (b) with the nucleic acid sequence of said desired MAPK pathway member; and (d) introducing to said desired MAPK pathway member a mutation equivalent to the mutation in said activated MAPK provided in (a).

The invention also relates to a constitutively activated mutant of a eukaryotic MAPK pathway member produced by this particular method of the invention.

In yet another aspect, the invention relates to an expression vector comprising nucleic acid sequence coding for a constitutively activated mutant of a MAPK pathway member operably linked to a promoter, terminator, any additional control, promoting and/or regulatory elements and optionally a selectable marker and/or a tag sequence. The said activated mutant of a MAPK pathway member comprised in the expression vector is the constitutively activated MAPK of the invention.

The promoter may be any one of a constitutive or an inducible promoter.

In yet a further aspect, the invention relates to a host cell transfected or transformed with any of the expression vectors of the invention, which host cell may be a prokaryotic or eukaryotic cell, particularly a bacterial cell, yeast cell, an insect cell, a plant cell an avian cell or a mammalian cell.

Still further, the invention relates to a non-human transgenic organism carrying a naked DNA sequence or an expression vector comprising the same, said DNA sequence coding for a constitutively activated mutant of a MAPK pathway member, particularly a constitutively activated mutant of the invention.

In a further aspect, the invention relates to a recombinant protein comprising a constitutively activated mutant of a MAPK pathway member capable of activating said MAPK pathway independently of the presence of an upstream kinase. The MAPK pathway member may be MAPK and MAPKK. The recombinant proteins of the invention thus preferably comprise an activated mutant according to the invention.

In an additional aspect, the invention relates to a method of screening for a substance which is an inhibitor of a MAPK pathway, which method comprises the steps of (a) providing a mixture comprising a constitutively activated mutant of a MAPK pathway member or any functional fragment thereof; (b) contacting said mixture with a test substance under conditions suitable for activation of said MAPK pathway; (c) determining the effect of the test substance on an end-point indication, wherein said effect is indicative of inhibition of said MAPK pathway by the test substance.

In this substance-screening method, the constitutively activated mutant of a MAPK pathway member is an activated mutant of any one of MAPK and MAPKK, particularly a mutant of MAPK or MAPKK, according to the invention.

The said reaction mixture may be according to a specific embodiment, a cell mixture or a cell-free mixture.

Thus, the reaction mixture may comprise (a) a constitutively activated mutant of any one of MAPK and MAPKK or any fragments thereof, in accordance with the invention; (b) an interactor molecule which can interact with said activated mutant, wherein said interaction indicates activation of said MAPK pathway by the activated mutant; and (c) optionally further solutions, buffers and compounds which provide suitable conditions for activation of said MAPK pathway and the detection of an end-point indication for the interaction of said activated mutant with said interactor molecule; whereby interaction of said interactor molecule with said activated mutant is detected by an end-point indication.

The interaction of the activated mutant with the interactor molecule in the presence of the test substance may be detected by the end-point indication, whereby inhibition of said end-point indicates inhibition of the MAPK pathway by said test substance.

The said reaction mixture may alternatively be a cell-free mixture.

The substance-screening method of the invention may thus employ said activated mutant or any fragments thereof, as a purified recombinant protein, as a fusion protein or as a cell lysate of a transformed host cell expressing said activated mutant, according to the invention.

The said interactor molecule may be a fusion protein comprising a downstream substrate of said MAPK pathway member. For example, the interactor molecule may be a GST fusion protein comprising a downstream substrate selected from the group consisting of GST-c-Jun, GST-ATF-2 or MBP.

The detection of the interaction of said interactor substrate molecule with said activated mutant, may be by detecting an enzymatic activity of said activated mutant, for example by a kinase assay.

In a particular embodiment, the invention relates to a method of screening for a substance which is an inhibitor of a MAPK pathway which comprises the steps of (a) providing a cell-free mixture comprising a constitutively activated mutant of a MAPK pathway member and a fusion protein of a respective downstream substrate of said MAPK pathway member; (b) contacting said mixture with a test substance under conditions suitable for an in vitro kinase assay; (c) determining the effect of the test substance on phosphorylation of said substrate by the activated mutant as an end-point indication, whereby inhibition of the phosphorylation indicates inhibition of said MAPK pathway by the test substance.

Alternatively, the reaction mixture may be a recombinant cell mixture.

In a particular embodiment, the invention relates to a method of screening for a substance which is an inhibitor of a MAPK pathway which method comprises the steps of (a) providing recombinant cell mixture comprising a constitutively activated mutant of a MAPK pathway member or any functional fragments thereof and an interactor molecule as described above; (b) contacting said recombinant cell mixture with a test substance; (c) detecting interaction of the activated mutant with the interactor molecule in the presence of the test substance by searching for an end-point indication, whereby inhibition of said end-point indicates inhibition of the MAPK pathway by said test substance.

The recombinant cell may be transformed or transfected by an expression vector according to the invention, coding for an active mutant of a MAPK pathway member according to the invention; and in addition by a construct comprising an interactor molecule which comprises transcriptional regulatory sequence and an operably linked reporter gene and optionally, further endogenously or exogenously expressible interactor molecules essential for activation of said pathway.

The interaction of said mutant of MAPK pathway member with downstream signaling molecules, results in mediation of transcription of said reporter gene, wherein the transcription is driven by said regulatory sequence.

The end-point indication may be the expression of said reporter gene, which leads to a visually detectable signal.

The activated mutant mediates transcription of the reporter gene leading to a detectable signal, whereby decrease of said detectable signal in the presence of the test substance indicates inhibition of the MAPK pathway by said test substance.

A recombinant cell, which is transformed with an expression vector coding for the activated mutant of a MAPK pathway member of the invention may further contain an endogenously or exogenously expressible interactor molecule essential for activation of said pathway.

Thus, the invention also relates to a method of screening for a substance which is an inhibitor of a MAPK which comprises the steps of (a) providing recombinant cell mixture comprising a constitutively activated mutant of a MAPK pathway member and downstream interactor molecules essential for transducing a signal through said pathway; (b) contacting said recombinant cell mixture with a test substance; and (c) determining the effect of said test substance on the interaction of the active mutant with the interactor molecules by detecting the end-point indication, wherein said effect is indicative of inhibition of the MAPK pathway by said test substance. The end-point indication may be a cell phenotype caused by activation of said MAPK pathway.

The activated mutant may optionally be expressed under an inducible promoter.

The recombinant cell is preferably a yeast cell, and the end-point indication may be the ability of the cells to proliferate, to survive under high osmotic conditions, to mate, to invade agar and/or to form pseudohyphae, to sporulate or any combinations thereof.

In yet another embodiment, the recombinant cell may also be a plant cell, insect cell, an avian cell or a mammalian cell, and the end-point indication may be the ability to induce apoptosis, ability to induce growth arrest, ability to induce oncogenic phenotype or the ability to induce differentiation.

The test substance to be screened by the various embodiments of the substance-screening method of the invention may be selected from the group consisting of: protein based, carbohydrates based, lipid based, nucleic acid based, natural organic based, synthetically derived organic based, antibody based, inorganic based and peptidomimetics based substances.

More specifically, said protein based substance may be a product of any one of positional scanning of peptide libraries, libraries of cyclic peptidomimetics, peptide combinatorial libraries and phage display random or dedicated libraries.

The test substance is preferably an inhibitor of said MAPK pathway.

The invention further relates to a method of preparing a therapeutic composition for the inhibition of a MAPK pathway in a mammalian subject in need of such treatment, which method comprises the steps of:

(a) identifying an inhibitor of a MAPK pathway; and

(b) admixing said inhibitor substance with at least one of a pharmaceutically acceptable carrier, diluent, excipient and/or additive.

In a particular embodiment, the inhibitor substance is identified by the screening method of the invention.

This therapeutic composition is intended for the treatment of a pathological disorder selected from the group consisting of: neoplasia, cancer, inflammation, degenerative diseases and immunological disorders.

The invention will be described in more detail on hand of the following drawings.

### BRIEF DESCRIPTION OF THE FIGURES

FIG. 1—Genetic screen of the invention

Figure shows a general scheme and rationale of the genetic screen of the invention. The strategy of said screening method comprises: (a) random, saturated mutagenesis of HOG) gene in vitro; (b) introducing the library of HOG1 mutants to pbs2Δ strain and selecting colonies on 1.1 M NaCl; (c) positive colonies should harbor active Hog1 variants which are independent of Pbs2 (MAPKK) activity. Abbreviations: casc. (cascade), str. (strain), Surv. In. Osm. (survive high osmolarity), Gr. (growth), N. gr. (no growth), asterisk indicates a constitutively active enzyme.

FIGS. 2A-2B—HOG1 active mutants are able to rescue pbs2Δ cells and hog1Δpbs2Δ cells from high salt concentrations HOG1 active mutants, but not HOG1 wild type, are able to rescue pbs2Δ cells and hog1Δpbs2Δ cells from high salt concentrations.

2A. On the left hand side are the two master plates supplemented with optimal growth medium, each plate is divided into two parts—the right side contains hog1Δ cells (used as controls [Schuller et al. 1994)]) and the left side contains pbs2Δ cells [Brewster et al. (1993)].

To the right of the master plates are the replica plates supplemented with 2 different concentrations of NaCl. PBS2 and. vec. (vector) written in white on the plates denote the plasmid identity in the cells of the upper row. In the rows below same plasmids (wt, HOG1, or mutants)-are expressed in both pbs2Δ and hog1Δ cells.

2B. Upper panel shows the master plate on the left. Plasmids are indicated in the scheme on the right; lower panel shows replica plating on two different salt concentrations.

Abbreviations: Plas. (plasmid), str. (strain), med. (media).

FIGS. 3A-3B—Hog1 activating mutations occurred in conserved amino acids within the corresponding MAPKs

Many of the Hog1 activating mutations occurred in amino acids, which are conserved in other MAPKs.

3A. The Hog1 amino acid sequence, sites of mutation of the invention are bold;

3B. Sequence alignment of yeast and mammalian MAPKs, showing mutation sites in bold. Phosphoacceptors Thr and Tyr are indicated by asterisk. Note that numbers in these sequences do not reflect actual locations of amino acids in the proteins but the alignment positions. Abbreviations: hum. (human), mou. (mouse), ra. (rat).

FIG. 4—Active Hog1 variants expressed in pbs2Δ cells activate the authentic Hog1 pathway in vivo, independent of Pbs2 Primer extension analysis [Engelberg et al. (1994)] of RNAs prepared from pbs2Δ cells expressing Hog1 mutants. Specific primers for GPD1, GPP2 and HAL3 were used [Stanhill et al. (1999)]. HAL3 was used as an internal control for mRNA amounts. Abbreviations: str. (strain), vec. (vector).

FIG. 5—Active Hog1 variants expressed in hog1Δ cells activate the authentic Hog1 pathway in vivo, independent of salt stimulation

Primer extension analysis of RNA prepared from hog1Δ cells expressing Hog1 mutants. Specific primers for GPD1 and GPP2 were used as described in the Examples. Abbreviations: str. (strain), vec. (vector).

FIGS. 6A-6E—Active Hog1 variants strongly activate the authentic Hog1 pathway in vivo, independent of salt stimulation, and induce feed back suppression of the pathway when constitutively expressed

Shown are the results of quantitation of Northern blot analysis challenged with different probes as indicated, blots were quantitated using phosphor-imager.

6A-6B. Hog1 molecules were expressed using a constitutive promoter, RNA samples were prepared at the indicated time points from cells harboring empty plasmids or with the indicated Hog1 variants. From each strain one sample was incubated in the presence or the absence of salt.

6A. Shows GPD1 gene expression in the presence of 1M NaCl.

6B. Shows GRE2 gene expression in the presence of 1M NaCl.

6C-6E. Similar analysis was performed in cells expressing Hog1 using the MET3 inducible promoter. RNA samples were prepared from the indicated cells at the indicated time points. Time zero is the time when methionine was removed from the growth media (and thereby induction of expression of Hog1 molecules) in one culture of each strain. An identical culture was continued to grow in the presence of methionine.

6C. Shows GPD1 gene expression under methionine induction.

6D. Shows GRE2 gene expression under methionine induction.

6E. Shows STL1 gene expression under methionine induction. Abbreviations; Ind. (induction), T (time), Act. (active), met (methionine), TT (wild type), S. Act. (super active).

FIGS. 7A-7B-Many active forms of Hog1 do not require phosphorylation for their activity

Western blot analyses showing the phosphorylation state of the different Hog1 mutants expressed in pbs2Δ cells or in hog1Δ cells. All Hog1 proteins used are fused to a HA epitope. The upper panel of each image shows the levels of dually phosphorylated Hog1 using anti phosph-p38 antibodies; the lower panel shows total Hog1 protein levels using anti-HA antibodies.

7A. Shows phosphorylation state of the different Hog1 mutants expressed in pbs2Δ cells.

7B. Shows phosphorylation state of the different Hog1 mutants expressed in hog1Δ cells.

Abbreviations: str. (strain) and vec. (vector).

FIGS. 8A-8B—Active Hog1 mutants can rescue yeast from high salt concentrations even if Tyr176 is converted to Phe

Active Hog1 mutants containing the mutation Y176F can rescue yeast from high salt concentrations, but cannot when containing the T174A mutation. Each plate is divided in two. Hog1 wild type and mutants on the left side contain the mutation T174A, and on the right side they contain the mutation Y176F.

8A. Hog1 molecules were expressed in hog1Δ cells. Positive controls (first row, left side) is a wild type Hog1 expressed in hog1Δ cells; negative control (first row, right side) is a wild type Hog1 expressed in pbs2Δ cells.

8B. Hog1 molecules were expressed in pbs2Δ cells. Positive control is Hog1^{F318S} expressed in pbs2Δ cells (first row, right side); negative control is wild type Hog1 expressed in pbs2Δ cells (first row, left side).

Abbreviations; con. (control), mast. Pla. (master plates), Str. (strain), Repl. (replicas).

FIG. 9A-9B—The role of both phosphorylation sites in Hog1 activity in both pbs2Δ cells

Elimination of both phosphorylation sites (indicated by two asterisks) in Hog1 wild type and mutants abolishes their activity in both pbs2Δ cells and in hog1Δ cells. Replacement of only Tyr176 with Phe does not abolish the activity of Hog1^{D170A} (indicated with one asterisk; A, left plate, row 5). Upper plates are master plates containing YNB-URA media, which were replica plated onto YPD+1.3M NaCl plates.

9A. Shows expression of Hog1 wild type and mutants in pbs2Δ cells.

9B. Shows expression of Hog1 wild type and mutants in hog1Δ cells. Abbreviations; mast. Pla. (master plates), str. (strain), Repl. (replicas).

FIGS. 10A-10C—Hog1 mutants catalytically activity

Hog1 wild type or active Hog1 mutants were expressed and immuno-precipitated from hog1Δ cells or pbs2Δ cells and assayed in vitro using GST-ATF2 as a substrate. Cells were either exposed or not exposed to the presence of 1M NaCl. Each upper panel shows the autoradiogram of the in vitro kinase assay.

Each lower panel in FIGS. 10A and 10B, shows Western blots of the lysates, displaying the Hog1 protein levels. A negative control for the western blot is a lysate prepared from hog1Δ cells harboring an empty vector (first two lanes of each lower panel from the left).

10A. Shows catalytic activity as indicated by in vitro kinase assay of wild type Hog1 and mutants that were expressed in hog1Δ cells. Negative control in this panel is Hog1 activity measured in pbs2Δ cells (left lanes).

10B. Shows catalytic activity as indicated by in vitro kinase assay of wild type Hog1 and mutants that were expressed in pbs2Δ cells. Note that wild type Hog1 shows no activity in pbs2Δ cells. Positive control in these cells is the activity of wild type Hog1 expressed in and immuno-precipitated from hog1Δ cells (first two lanes of each panel from the left).

10C. Shows similar in vitro kinase assay performed on wild type Hog1 and on active mutants carrying additional mutation of Tyr176 to Phe or Thr174 to Ala, which were expressed in hog1Δ cells. Note that mutating the Tyr176 to Phe or Thr174 to Ala in the wild type Hog1 totally abolished activity. Converting the Tyr176 to Phe in the activating mutants did not change activity. The upper panel shows the autoradiogram of the in vitro kinase assay. The middle panel shows western blots of the lysates, displaying the phosphorylated threonine (Thr.) levels, and the lower panel shows Western blots of the lysates, displaying the Hog1 protein levels.

Abbreviations; str. (strain), vec. (vector), cont. (control of empty vector), GST (Glutathion —S— transferase).

FIGS. 11A-11B—Mutated human p38 harboring mutations equivalent to mutations identified in Hog1 are active as recombinant molecules Human p38 proteins carrying mutations equivalent to those that activate Hog1 possess an intrinsic catalytic activity and the capability to autophosphorylate. Wild type p38 protein as well as the mutants p38^{F327L} and p38^{F327S}, were expressed in E. coli and purified to near homogeneity. Purified proteins were subjected to in vitro kinase assay. Assay mixtures were separated through SDS-PAGE. Gels were stained with Coomassie brilliant blue (upper panels) and exposed to X-ray film (lower panels).

11A. Shows in vitro kinase assay using GST-ATF2 as a substrate.

11B. Shows autophosphorylation activity of the different mutants. Abbreviations: kina. Ass. (kinase assay), hum. (human), coom. (coomassie), WT (wild type), Rad (radioactivity), Au-Ph (auto phosphorylation).

FIGS. 12A-12B—p38 mutants are hyperactive in mammalian cells in culture

The indicated p38 activated mutants or p38 wild type genes were introduced into COS-7 cells in duplicates. Forty eight hours after transfection one set of plates was exposed to UV radiation. One hour later, protein lysates were prepared, transfected p38 molecules were immunoprecipitated and assayed in a kinase assay using GST-ATF2 as a substrates. Incorporation of radioactive phosphates to the substrate were quantitated using a phosphoimager. To calculate the fold induction, the level of incorporation in the assay with the wild type molecule (in the cells that were not exposed to UV radiation) was set as 1.

12A. Shows overall activation of p38 mutants that were either stimulated or not with UV, indicated as folds of activation.

12B. Compares activation of wild type p38 to activation of mutants in the absence of UV stimulation.

Abbreviations: F. Act. (folds of activation), ind. (induction), UV (ultra violet), WT (wild type), UV. (ultra violet).

FIG. 13—Combination of two mutations in the same MAPK increases activity

13A. illustrates the construction of doubly mutated molecules, the schematic lines showing HOG1 DNA harboring two mutations each (indicated by asterisks and by location (above). Phosphoacceptors Thr and Tyr are also indicated by asterisks.

13B. shows Western blot analysis demonstrating the expression of the double mutants using the MET3 inducible expression system.

13C. Expression of doubly mutated Hog1 induces growth arrest. The indicated Hog1 variants were introduced to the yeast strain JBY13 on a MET3 based plasmids. In the presence of methionine in the medium (upper plate) all strains grew normally. On a plate that did not contain methionine (and Hog1 molecules were expressed) cells harboring Hog1^{D170AF318L} and Hog1^{D170AF318S} could not give rise to single colonies (bottom two rows).

Abbreviations: Met (methionine), Vec. (vector), Con. (control of empty vector), phosph. Sit. (phosphorylation site), hrs. (hours), WT (wild type).

### DETAILED DESCRIPTION OF THE INVENTION

Although MAPKs are involved in pivotal biological processes and are therefore extensively studied, it has been difficult to address the exact role of a given MAPK in a particular biological system. Such a question could be approached using constitutively active forms of MAPKs, which were hitherto not available. This application describes a genetic screen in yeast that provides MAPK molecules which function independently of their MAPKK. The basic rationale behind this screen is that only an active form of a MAPK would induce the appropriate respective phenotype in a MAPKK null strain. This rationale was applied in the present invention for isolation of active Hog1 molecules. The mutants isolated could execute all Hog1 biochemical and biological activities independent of its respective MAPKK, Pbs2 activity and of the activity of any other MAPKK. Furthermore, the active Hog1 variants affected dramatically growth rate and other properties of the cell, exhibiting novel biological activities of Hog1. Their ability to perform all these functions stems from the very strong intrinsic catalytic activity they acquired.

Thus, in a first aspect, the present invention relates to a method of screening for a constitutively activated mutant of a desired eukaryotic MAPK pathway member of a MAPK pathway, which method comprises the steps of:

(a) providing a mutant yeast strain devoid of an upstream kinase, which kinase normally activate the MAPK pathway member in a non-mutant strain of said yeast, but can activates any equivalent or corresponding eukaryotic MAPK pathway member;

(b) obtaining a DNA library of various different mutants of a mutagenized gene coding for said desired MAPK pathway member, which library is capable of being expressed by a yeast cell;

(c) introducing said library into said deficient yeast strain under conditions suitable for activation of the yeast MAPK pathway;

(d) detecting an end-point indication for activation of the yeast MAPK pathway, which activation results from rescuing said pathway by a constitutively activated mutant of the desired MAPK pathway member; and optionally

(e) isolating said constitutively activated mutant from selected rescued clones.

It is to be appreciated that two possibilities are acceptable with regard to step (c). Firstly, it is possible to introduce the mutation library into a yeast and only then to provide suitable conditions for activation of said pathway. Another possibility is to introduce the library under condition suitable for the activation of said pathway, and thus providing immediate conditions for activation.

In one embodiment, activation of such constitutively activated mutant is independent of the presence of a kinase upstream thereto. This upstream kinase is capable of directly phosphorylating and activating said MAPK pathway member. It is to be appreciated that the active mutant produced by the method of the invention is capable of activating the authentic pathway downstream.

In a specific preferred embodiment, the method of the invention utilizes any yeast strain. As a non-limiting example, such yeast may be selected from the group consisting of Saccharomyces cerevisiae and Schizosaccharomyces pombe. Preferably, the selected yeast is Saccharomyces cerevisiae.

In a specifically preferred embodiment, the end-point indication in step (d) of the screening method of the invention may be the ability of the cells to survive under high osmotic conditions, ability to mate, ability to form invasive growth and pseudohyphae, the ability of the yeast to sporulate or any combinations thereof.

In a particularly preferred embodiment, the method of the invention is intended for screening for any activated eukaryotic MAPK or MAPKK. Such eukaryotic MAPK pathway member is selected from the group consisting of mammalian, plant cells, insect cells, avian cells, fungi cells and yeast cell MAPKs or MAPKKs.

The rationale that was successfully applied herein for isolation of active Hog1 molecules could be used to screen for other active MAPKs. For example, active forms of Fus3 could be isolated in cells lacking the MAPKK Ste7, using the mating phenotype as the biological assay. Analogously, constitutively active forms of Kss1 could induce a pseudohyphae phenotype in ste7Δ diploid strains. Furthermore, one can envisage a screen for active forms of mammalian MAPKs in yeast. For example, as p38 and JNK are biologically functional in yeast [Galcheva-Gargova et al. (1994); Han et al., (1994)], it should be possible to screen for active forms of these enzymes directly in pbs2Δ cells. However, as indicated by Example 6, insertion of mutations equivalent to the mutations found in Hog1, into the human p38α molecule, resulted in active p38 mutant (as demonstrated by FIGS. 11 and 12). Thus, screening for active forms of other MAPKs may not be necessary and insertion of respective mutation to a respective kinase may be sufficient, as will be described in a further particular method of the invention herein after. The activating mutations identified in Hog1 could be applied directly in many other MAPKs.

Therefore, as a specifically preferred embodiment, the invention relates to a method of screening for an activated mutant of MAPK. Such MAPK may be selected for example, from the group consisting of plant cells, insect cells, avian cells or mammalian ERK1, ERK2, ERK 5, ERK 7, JNK, p38 subfamilies, the yeast and fungi Fus3, Kss1, Mpk1 and Hog1.

Thus, in a particular embodiment, the MAPK may be any one of plant, avian, insect, or mammalian ERK subfamily, or the yeast and fungi Fus3, Kss1 and Mpk1. In this specific example the method of the invention comprises the steps of:

(a) providing the mutant yeast strain ste7Δ devoid of the MAPKK, Ste7, which is upstream kinase of any one of Fus3 and Kss 1;

(b) providing a DNA library of different mutants of a mutagenized gene coding for said MAPK, which MAPK may be one of fungi, yeast, plant, insect, avian and mammalian MAPKs;

(c) introducing said library into said ste 7Δ yeast strain under conditions suitable for activation of said yeast MAPK pathway;

(d) detecting an end-point indication for activation of the yeast MAPK pathway, which activation results from rescuing said pathway by a constitutively activated mutant of said MAPK; and optionally

(e) isolating said constitutively activated MAPK mutant from selected positive rescued clones.

In one specifically preferred embodiment the yeast MAPK pathway utilized is the Ste7/Fus3 and the end-point indication is the ability of rescued cells to mate, or the Mkk/Mpk1 pathway with the end-point indication being the ability to rescue cells from hypoosmotic conditions. Alternatively, the yeast MAPK pathway may be the Ste7/Kss1 pathway, and the end-point indication may be the ability of rescued cells to form invasive growth and pseudohyphae.

In another preferred embodiment the invention relates to a method of screening for MAPK such as plant, insect, avian or mammalian JNK or p38 subfamily members, and the yeast Hog1. In this specific example the method comprises the steps of:

(a) providing the mutant yeast strain pbs2Δ devoid of the respective upstream MAPKK, Pbs2, which Pbs2 is a kinase upstream to Hog1 and is capable of directly phosphorylates and activates Hog1;

(b) providing a DNA library of different mutants of a mutagenized gene coding for said MAPK, which may be a yeast, fungi, plant, insect, avian or mammalian MAPK;

(c) introducing said library into said pbs2Δ yeast strain under conditions suitable activation of said yeast MAPK pathway;

(d) detecting an end-point indication for activation of the yeast MAPK pathway, which activation results from rescuing of said pathway by a constitutively activated mutant of said MAPK; and optionally

(e) isolating said constitutively activated MAPK mutant from said selected rescued clones.

It is to be noted that in this particular embodiment, Hog1 may serve as the MAPK equivalent to the desired MAPK (that may be selected from ERK, JNK and p38 subfamilies). Therefore, the method described in this embodiment is useful for screening for Hog1 mutants as well as for MAPKs which are equivalent to Hog1.

In such example, the yeast MAPK pathway may be the Pbs2/Hog1 and the end-point indication may be the ability of rescued cells to survive under high osmotic conditions.

A specifically preferred embodiment relates to the yeast MAPK, Hog1. In such specific embodiment the method of the invention comprises the steps of:

(a) providing the mutant yeast strain pbs2Δ devoid of the upstream MAPKK, Pbs2;

(b) providing a DNA library of different mutants of a mutagenized HOG1 gene coding for the MAPK Hog1;

(c) introducing said library of mutants into said pbs2Δ yeast strain under conditions suitable for activation of said yeast Pbs2/Hog1 pathway, which conditions include, for example highly osmotic growth medium;

(d) detecting an end-point indication for activation of the yeast Pbs2/Hog1 pathway, which activation results from rescuing said pathway by a constitutively activated Hog1 mutant; and optionally

(e) isolating said constitutively activated Hog1 mutant from selected rescued clones.

Such constitutively activated Hog1 mutant is capable of activating the authentic pathway downstream to Hog1 independently of Pbs2 and endogenous Hog1, as also demonstrated by Example 2.

In a specifically preferred embodiment, the mutant obtained by the method of the invention carries at least one mutation in the conserved L16 domain of any MAPK. Preferably, mutation within the L16 domain is between residues 314 to 332, as located within the Hog1 amino acid sequence as denoted by SEQ ID No. 1, or alternatively, in the L16 domain of any MAPK. This mutant has at least one mutation selected from the group consisting of point mutations, missense, nonsense, insertions, deletions or rearrangement.

In addition to suggesting particular mutations that may render MAPKs constitutively active, the mutations identified in the present invention point at the domains that should be mutated in an effort to activate MAPKs. Six of the nine mutations were found in L16 between residues 314 to 332. This domain is equivalent to residues 323 to 341 in ERK2, which has an important role in forming the interface for dimerization [Khokhlatchev et al. (1998)]. It is likely that the activating mutations (in particular mutations that replac a charged amino acid with a hydrophobic residue) support formation of intermolecular contacts and consequently the formation of an active dimer.

Further support for this notion comes from the D170A mutation. D170 is homologous to D173 in ERK2, which resides near H176 that forms an ion pair with E343 of another monomer [Khokhlatchev et al. (1998)]. The possibility that each mutant stabilizes a dimer suggests that combining several mutations on the same MAPK molecule would generate an even more stable and more active dimer. Another possibility to explain the intrinsic activity of the mutants is that the mutations strengthen the contacts between L16 and the phosphorylation lip of the same molecule. Such contacts may result in refolding the phosphorylation lip from the closed conformation to the open structure [Canagarajah et al. (1997)].

In a specifically preferred embodiment, said mutant has at least one point mutation in the L16 domain located at any position of A314, F318, W320, F322, W332 or mutation located out of the L16 domain at positions such as Y68, D170, N391 and any combinations thereof. Most preferred mutants according to a particular embodiment may carry at least one mutation selected from the group consisting of A314T, F318L, F318S, W320R, F322L, W332R or mutation out of the L16 domain such as Y68H, D170A, N391D and any combinations thereof. These particular numerical locations refer to the Hog1 amino acid sequence as denoted by SEQ ID No. 1. However, it is to be appreciated that the specific location of these mutations is contemplated to be within the scope of the present invention. Moreover, any other mutations occurring in these sites are also within the scope of the invention (e.g. W320 may preferably mutated to R or to any other amino acid residue).

Still further, it is to be understood that any equivalent mutations in any corresponding MAPK are contemplated to be within the scope of the invention. These particular equivalents are detailed hereinafter. More specifically, these equivalents may be selected from ERK, JNK and p38 subfamilies members, as also exemplified by FIG. 3.

By a “corresponding MAPK” or “equivalent MAPK” as used herein is meant a MAPK pathway member of any organism origin located in a corresponding location in the MAPK signal transduction pathway of said organism. For example, mammalian ERK, JNK and p38 are MAPK and therefore are positioned in the mammalian MAPK pathway in a corresponding location to that of Hog1 within the Pbs2/Hog1 pathway.

According to another embodiment the method of the invention may be utilized for screening for an activated MAPKK.

According to a specific embodiment, such MAPKK may be selected from the group consisting of plant, insect, avian or mammalian MEKK, JNKK, the yeast and fungi Ste7 and Byr1, which method comprises the steps of:

(a) providing a mutant yeast strain MAPKKKA devoid of the kinase upstream to said MAPKK, which is MAPKKK;

(b) obtaining a DNA library of different mutants of a mutagenized gene coding for said MAPKK, which may be a yeast, fungi, plant, insect, avian or mammalian MAPKK;

(c) introducing said library into said MAPKKKA yeast strain under conditions suitable for activation of said yeast MAPK pathway;

(d) detecting an end-point indication for activation of the yeast MAPK pathway which activation results from rescuing said pathway by a constitutively activated mutant of the MAPKK; and optionally

(e) isolating said constitutively activated MAPKK mutant from selected rescued clones.

In a second aspect, the present invention relates to constitutively activated mutant of a MAPK pathway member, capable of activating said MAPK pathway independently of the kinase upstream thereto. The mutant of the invention has at least one mutation selected from the group consisting of point mutation, missense nonsense, insertion, deletion or rearrangement.

According to one embodiment of said aspect, the MAPK pathway member may be any one of MAPK and MAPKK. More particularly said MAPK pathway member is a eukaryotic MAPK pathway member selected from the group consisting of mammalian, plant cells, insect cells, avian cells fungi cells and yeast cells MAPK pathway member.

In a preferred example the activated mutant of the invention is a MAPK. More specifically, said MAPK may be selected from the group consisting of mammalian ERK, ERK5, ERK7, JNK, p38 subfamilies, the yeast and fungi Fus3, Kss1, Mpk1 and Hog1.

A particular Hog1 mutant is capable of activating the authentic pathway downstream to Hog1 independently of Pbs2 and endogenous Hog1.

In yet another embodiment, the activated MAPK mutant of the invention may have at least one mutation occurring in the conserved L16 domain of the Hog1 protein, particularly between residues 314 to 332 of said L16 domain as located in the amino acid sequence Hog1 as denoted by SEQ ID No. 1. In case the mutant of the invention is a MAPK equivalent to Hog1, such as for example ERK, JNK and p38 family members, a preferred mutant may carry at least one point mutation in the respective L16 domain of any of said MAPKs.

These activated MAPK mutants may have at least one point mutation in Hog1 or in a respective mutation in any MAPK, which mutation may be located at any position of A314, F318, W320, F322, W332 and any combinations thereof. These numerical locations refer to the Hog1 amino acid sequence as denoted by SEQ ID No. 1. Preferred mutants may have at least one point mutation selected from A314T, F318L, F318S, W320R, F322L, W332R and any combinations thereof. However, where the MAPK mutant is a Hog1 equivalent molecule, these specific mutations should be located within the corresponding sequences of these MAPK molecules. Such mutations, whether in HOG1 or other gene, are referred to as respective mutations.

In a particular embodiment, the Hog1 mutant of the invention may carry a point mutation selected from A314T, F318L, F318S, W320R, F322L, W332R mutations as located in the Hog1 amino acid sequence, and any combination thereof. In the activated MAPK mutants of the invention the said respective mutation in any MAPK may be located at position A342 or F346 of the human ERK1, A325 or F329 of the human ERK2, A323 or A327 of the rat ERK2, F362 and F366L of the human ERK5, W352 of the human JNK1 and A320, F327, or W337R of the rat and human p38 and any combination thereof. Most preferred mutants may carry a point mutation selected from the group consisting of A342T, F346L and F346S of the human ERK1, A325T, F329S and F329L of the human ERK2, A323T, A327L and A327S of the rat ERK2, F362L, F362S and F366L of the human ERK5, W352R of the human JNK1 and A320T, F327L, F327S, W337R of the rat and human p38 and any combination thereof, particularly W337R, F327S and F327L that are point mutations in the human p38. These numerical locations refer to the human ERK1, human and rat ERK2, the human ERK5, the human JNK1 and the human and rat p38 amino acid sequences, as denoted by the GenBank accession numbers P27361, P28482, M64300, U25278, P45983, L35253 and AF346293, respectively. These sequences are fully incorporated herein by reference.

Alternatively, the activated mutants of the invention may carry at least one point mutation in Hog1 or a respective mutation in any MAPK, which is located out of the L16 domain. This mutation may be located at position Y68, D170 and N391, or in corresponding location in any other equivalent MAPK. More particularly, the activated mutants of the invention may carry at least one point mutation selected from the group consisting of Y68H, D170A and N391D, or in corresponding location in any other equivalent MAPK. More particularly, wherein the mutant is a Hog1 mutant, the point mutation may be selected from the group consisting of Y68H, D170A, N391D and any combinations thereof, as referred to the Hog1 amino acid sequence (SEQ ID No. 1).

In the activated MAPK mutants of the invention, the said respective mutation in any of said MAPK may be located at any position of D192 of the human ERK1, D175 of the human ERK2, D173 of the rat ERK2, Y71 of the human JNK1, Y68 and D176 of the mammalian p38. These locations refer to the human ERK1, the human ERK2 the rat ERK2, the human JNK1 and the human or the rat p38 amino acid sequences as denoted by the GenBank accession numbers P27361, P28482, M64300, L35253 and AF346293, respectively, and fully incorporated herein by reference.

Preferred mutants of respective MAPK may be selected from the group consisting of D192A of the human ERK1, D175A of the human ERK2, D173A of the rat ERK2, Y71H of the human JNK1, Y68H and D176A of the mammalian p38.

In a particular embodiment, the invention relates to an active mutant of p38. This mutant carries at least one point mutation located at position Y68, D176, W320, W337 and F327, or any combinations thereof. Most preferred mutant may be any one of Y68H, D176A, W320T, W337R, F327S and F327L, or any combinations thereof. These mutations are numbered according to the human p38 amino acid sequence of GeneBank accession no. L35253 fully incorporated herein by reference.

Moreover, mutations of any particular preferred sites are not limited to the amino acid residues that were isolated using the screening method of the invention. Such particular sites in Hog1 or in any corresponding MAPK may be mutated to any amino acid residue.

It should be also understood that any of the mutants of the invention may further carry additional mutations, such as mutations of the phosphoacceptors sites, for example, Thr 174 and Tyr 176 of Hog1, as described in Example 4.

The invention also relates to an activated MAPK or MAPKK obtained by the method of the invention.

Moreover, the active mutant of the invention may carry at least one point mutation located at the preferred positions described by the invention. Any mutant of the invention may comprise more than one mutation. As non-limiting example, a preferred mutant may carry about one to nine mutation, more preferably, one to six mutations. Most preferred, are active mutants carrying two mutations at the preferred positions of the invention. Non-limiting examples for such particular mutants are the double mutants described in Example 7.

It is possible that more than one mechanism underlies the activity of the mutants. Some mutants may form spontaneous dimers while others could use another mechanism. The differences in the intrinsic catalytic activities of the various mutants in fact suggest different mechanisms. Structural studies may reveal the changes in protein folding induced by each mutation. Without being bound by any theory, it may be suggested that combining, on the same Hog1 molecule, a mutation that uses one mechanism with a mutation that uses another, may generate an enzyme molecule that is even more active in vivo than the molecules described herein. As exemplified by Example 7, combination of activating mutations in Hog1 molecule, dramatically increases the biological effect of the mutants. The invention therefore, further relates as a particular MAPK mutant to any of the double mutants described in FIG. 13A and in Example 7.

The general importance of the mutants identified is manifested by the fact that insertion of similar mutations to the human p38α kinase rendered it catalytically active.

The results with p38α open the way to producing more active forms of various MAPKs based on the mutations identified in Hog1. Insertion of appropriate mutations to JNK [equivalent to the HOG) mutations Y68H, and W322R (FIG. 3)] and to the ERK2 [equivalent to the HOG1 mutations D170A, A314T, F318S and F318L (FIG. 3), as well as to the equivalent sites in ERK1 and ERK5], may render these MAPKs active. Such mutations in a variety of MAPKs could provide a useful battery of active forms of each MAPK.

Therefore, as a further aspect the invention relates to a method of producing a desired constitutively activated mutant of a eukaryotic MAPK pathway member, which method comprises the steps of (a) providing an activated MAPK pathway member of the invention; (b) subjecting the activated mutant provided in step (a) to nucleic acid sequence analysis; (c) aligning the sequence obtained in step (b) with the nucleic acid sequence of said desired MAPK pathway member; and (d) introducing to said desired MAPK pathway member a mutation equivalent to the mutation in said activated MAPK provided in (a). Such method was specifically employed in creation of human p38 mutants, as demonstrated by Example 6.

The invention also relates to a constitutively active mutant of a eukaryotic MAPK pathway member produced by this particular method of the invention. For example, an active mutant of p38 that carries at least one point mutation located at positions W337, F327, or any combinations thereof. Preferably, at least one point mutation of W337R, F327S and F327L or any combinations thereof. These mutations are numbered according to the human p38 amino acid sequence of GeneBank accession no. L35253 which is fully incorporated herein by reference.

It is to be understood that the invention further relates to the nucleic acid sequences coding for any of the mutants of the invention, as well as to any of the mutants isolated using the method of the invention.

In yet another aspect, the invention relates to an expression vector comprising a nucleic acid sequence coding for a constitutively activated mutant of a MAPK pathway member operably linked to a promoter, terminator, any additional control, promoting and/or regulatory elements and optionally a selectable marker and/or a tag sequence. The said activated mutant of a MAPK pathway member comprised in the expression vector may be any one of the constitutively activated MAPKs of the invention.

“Expression Vehicles”, as used herein, encompass vectors such as plasmids, viruses, bacteriophage, integratable DNA fragments, and other vehicles, which enable the integration of DNA fragments into the genome of the host. Expression vectors are typically self-replicating DNA or RNA constructs containing the desired gene or its fragments, and operably linked genetic control elements that are recognized in a suitable host cell and effect expression of the desired genes. These control elements are capable of effecting expression within a suitable host. Generally, the genetic control elements can include a prokaryotic promoter system or a eukaryotic promoter expression control system. Such system typically includes a transcriptional promoter, an optional operator to control the onset of transcription, transcription enhancers to elevate the level of RNA expression, a sequence that encodes a suitable ribosome binding site, RNA splice junctions, sequences that terminate transcription and translation and so forth. Expression vectors usually contain an origin of replication that allows the vector to replicate independently of the host cell.

Plasmids are the most commonly used form of vector but other forms of vectors which serves an equivalent function and which are, or become, known in the art are suitable for use herein. See, e.g., Pouwels et al. Cloning Vectors: a Laboratory Manual (1985 and supplements), Elsevier, N.Y.; and Rodriquez, et al. (eds.) Vectors: a Survey of Molecular Cloning Vectors and their Uses, Buttersworth, Boston, Mass (1988), which are incorporated herein by reference.

In general, such vectors contain in addition specific genes, which are capable of providing phenotypic selection in transformed cells. The use of prokaryotic and eukaryotic viral expression vectors to express the genes coding for the activated MAPK pathway members or of any fragments thereof, of the present invention are also contemplated.

The vector is introduced into a host cell by methods known to those of skilled in the art. Introduction of the vector into the host cell can be accomplished by any method that introduces the construct into the cell, including, for example, calcium phosphate precipitation, microinjection, electroporation or transformation. See, e.g., Current Protocols in Molecular Biology, Ausuble, F. M., ed., John Wiley & Sons, N.Y. (1989).

As used herein, the term “nucleic acid” refers to polynucleotides such as deoxyribonucleic acid (DNA), and, where appropriate, ribonucleic acid (RNA). The terms should also be understood to include, as equivalents, analogs of either RNA or DNA made from nucleotide analogs, and, as applicable to the embodiment being described, single-stranded (such as sense or antisense) and double-stranded polynucleotides The term “operably linked” is used herein for indicating that a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

Accordingly, the term control and regulatory elements includes promoters, terminators and other expression control elements. Such regulatory elements are described in Goeddel; [Goeddel., et al., Gene Expression Technology: Methods in Enzymology 185, Academic Press, San Diego, Calif. (1990)]. For instance, any of a wide variety of expression control sequences that control the expression of a DNA sequence when operatively linked to it may be used in these vectors to express DNA sequences encoding any desired active MAPK pathway member obtained by the method of this invention.

A variety of selectable markers can be incorporated into any construct. For example, a selectable marker which confers a selectable phenotype such as drug resistance, nutritional auxotrophy, resistance to a cytotoxic agent or expression of a surface protein, can be used.

Selectable marker genes which can be used include neo, gpt, dhfr, ada, pac, hyg, CAD, KAN, URA3, HIS3 and LEU2. The selectable phenotype conferred makes it possible to identify and isolate recipient cells. Amplifiable genes encoding selectable markers (e.g., ada, dhfr and the mutifunctional CAD gene which encodes carbamyl phosphate synthase, aspartate transcarbamylase, and dihydro-orotase) have the added characteristic that they enable the selection of cells containing amplified copies of the selectable marker inserted into the genome. This feature provides a mechanism for significantly increasing the copy number of an adjacent or linked gene for which amplification is desirable.

The expression vector of the invention may further comprise a tag sequence. Such sequences enable the detection and isolation of the recombinant protein. As a non limiting example such tag sequences may be any one of HA (hemaglutinine), c-myc, GST (glutathione-s-transferase), GFP (green fluorescent protein) and His-6 (six histidine residues).

The promoter is optionally an inducible promoter. Inducible promoters and inducible genetic elements are known in the art and can be derived from viral, yeast or mammalian genomes. Numerous examples of inducible promoters are known in the art. As a particular example, the expression vectors of the invention utilize the MET3 inducible system [Cherest., et al., (1984)]. As shown by Example 2, expression of the different mutants is induced only in the absence of methionine. Examples for other inducible promoters are metallothionein promoter, inducible by heavy metals [Mayo, et al., (1982)], the mouse mammary tumor virus (MMTV) promoter, induced by glucocorticoid [Beato, et al., (1987)], the TET promoter which is repressed by tetracycline [Pescini, et al., (1994)] and the lac repressor-lac operator inducible promoter system. This E. coli system, based on the DNA binding protein namely lac repressor (lacI), and the lac operator (lacO), has been shown to function in mammalian cells [Brown, et al., (1987)].

In yet a further aspect, the invention relates to a host cell transformed with any of the expression vectors of the invention, which host cell may be a prokaryotic or eukaryotic cell, particularly a bacterial cell, yeast cell, an insect cell, avian cell, a plant cell or a mammalian cell.

“Cells”, “host cells” or “recombinant cells” are terms used interchangeably herein. It is understood that such terms refer not only to the particular subject cells but to the progeny or potential progeny of such a cell. Because certain modification may occur in succeeding generation due to either mutation or environmental influences, such progeny may not, in fact, be identical to the parent cell, but are still included within the scope of the term as used herein.

“Host cell” as used herein refers to cells which can be recombinantly transformed with vectors constructed using recombinant DNA techniques. A drug resistance or other selectable marker is intended in part to facilitate the selection of the transformants. Additionally, the presence of a selectable marker, such as drug resistance marker may be of use in keeping contaminating microorganisms from multiplying in the culture medium. Such a pure culture of the transformed host cell would be obtained by culturing the cells under conditions which require the induced phenotype for survival.

As used herein, the term “transfection” means the introduction of a nucleic acid, e.g., an expression vector, into a recipient cells by nucleic acid-mediated gene transfer. “Transformation”, as used herein, refers to a process in which a cell's genotype is changed as a result of the cellular uptake of exogenous DNA or RNA, and, for example, the transformed cell expresses the activated MAPK mutants of the invention. It is to be appreciated that any other method known in the art for introducing DNA, RNA or protein (mutated protein molecules of the invention), may be used for expressing the activated mutants of the invention in any cell.

Still further, the invention relates to a non-human transgenic microorganism or organism carrying a DNA sequence or an expression vector comprising the same, said DNA sequence coding for a constitutively activated mutant of a MAPK pathway member, particularly a constitutively activated mutant of the invention.

As used herein, a “transgenic organism” is any microorganism or organism such as plant or animal, preferably a non human mammal, bird or an amphibian, in which one or more of the cells of the animal contain heterologous nucleic acid introduced by way of human intervention, such as by transgenic techniques well known in the art.

The nucleic acid is introduced into the cell, directly or indirectly by introduction into a precursor of the cells, by way of deliberate genetic manipulation, such as by microinjection or by infection with a recombinant virus. The term genetic manipulation does not include classical cross-breeding or in vitro fertilization, but rather is directed to the introduction of a recombinant DNA molecule. This molecule may be integrated within a chromosome, or it may be extrachromosomally replicating DNA. In the typical transgenic organisms described herein, the transgene causes cells to express a recombinant form of the activated mutant of the invention.

Transgenic organisms also include both constitutive and conditional “knock out” microorganisms, animals or plants. The “non-human organisms” of the invention include plants, vertebrates such as rodents, non-human primates, sheep, dog, cow, chickens, amphibians, reptiles, etc. Preferred non-human animals are selected from the rodent family including rat and mouse, most preferably mouse. The term “chimeric organism” is used herein to refer to an organism in which the recombinant gene is found, or in which the recombinant is expressed in some but not all cells of the animal.

In a further aspect, the invention relates to a recombinant protein comprising a constitutively active mutant of a MAPK pathway member.

This mutant is capable of activating said MAPK pathway independently of the presence of an upstream kinase. The MAPK pathway member may be MAPK and MAPKK. The recombinant proteins of the invention thus preferably comprise an activated mutant according to the invention.

In an additional aspect, the invention relates to a method of screening for a substance which is an inhibitor of a MAPK pathway, which method comprises the steps of (a) providing a mixture comprising a constitutively activated mutant of a MAPK pathway member or any functional fragments thereof; (b) contacting said mixture with a test substance under conditions which normally lead to activation of said MAPK pathway; (c) determining the effect of the test substance on an end-point indication, wherein said effect is indicative of inhibition of said MAPK pathway by the test substance.

In this substance-screening method, the constitutively active mutant of a MAPK pathway member is an activated mutant of any one of MAPK and MAPKK, particularly, a mutant of MAPK or MAPKK according to the invention.

The said reaction mixture may be particularly any one of a cell mixture or a cell-free mixture.

Thus, the reaction mixture may comprise (a) a constitutively activated mutant of any one of MAPK and MAPKK in accordance with the invention or any functional fragment thereof; (b) an interactor molecule which can interact with said activated mutant, wherein said interaction indicates activation of said MAPK pathway by the activated mutant; and (c) optionally further solutions, buffers and compounds which provide suitable conditions for activation of said MAPK pathway and the detection of an end-point indication for the interaction of said activated mutant with said interactor molecule; whereby interaction of said interactor molecule with said activated mutant is detected by an end-point indication.

The interaction of the activated mutant with the interactor molecule in the presence of the test substance may be detected by the end-point indication, whereby inhibition of said end-point indicates inhibition of the MAPK pathway by said test substance.

According to one embodiment, the reaction mixture may be a cell-free mixture.

The substance-screening method of the invention may thus employ said activated mutant or any functional fragment thereof, as a purified recombinant protein of the invention, or as a cell lysate of a transformed host cell according to the invention expressing said activated mutant.

The said interactor molecule may be a fusion protein comprising a downstream substrate of said MAPK pathway member. For example, the interactor molecule may be a GST fusion protein comprising a substrate selected from the group consisting of GST-c-Jun, GST-ATF-2 or MBP.

The detection of the interaction of said interactor substrate molecule with said activated mutant, may be by detecting an enzymatic activity of said activated mutant, for example by a kinase assay.

In a particular embodiment, the invention relates to a method of screening for a substance which is an inhibitor of a MAPK pathway which comprises the steps of (a) providing a cell free mixture comprising a constitutively activated mutant of a MAPK pathway member and a fusion protein of a respective downstream substrate of said MAPK pathway member; (b) contacting said mixture with a test substance under conditions suitable for an in vitro kinase assay; (c) determining the effect of the test substance on phosphorylation of said substrate by the activated mutant as an end-point indication, whereby inhibition of the phosphorylation indicates inhibition of said MAPK pathway by the test substance.

Alternatively, the reaction mixture may be a recombinant cell mixture.

Therefore, in a particular embodiment, the invention relates to a method of screening for a substance which is an inhibitor of a MAPK pathway which method comprises the steps of (a) providing recombinant cell mixture comprising a constitutively activated mutant of a MAPK pathway member and any fragment thereof, and an interactor molecule; (b) contacting said recombinant cell mixture with a test substance; (c) detecting interaction of the activated mutant with the interactor molecule in the presence of the test substance by searching for an end-point indication, whereby inhibition of said end-point indicates inhibition of the MAPK pathway by said test substance.

The recombinant cell may be transformed or transfected by an expression vector according to the invention, coding for an activated mutant of a MAPK pathway member according to the invention; and by an additional construct comprising an interactor molecule which comprises transcriptional regulatory sequence and an operably linked reporter gene.

As non-limiting examples, such reporter genes may be selected from the group consisting of green fluorescent protein (GFP), luciferase, secreted alkaline phosphatase (SEAP) and β-galactosidase (β-gal).

The said transcriptional regulatory sequence is sensitive to intracellular signals transduced by interaction of said MAPK pathway member with downstream signaling molecules mediating the transcription of said reporter gene, wherein the transcription being driven by said regulatory sequence.

The end-point indication may be the expression of said reporter gene, which leads to a visually detectable signal. The activated mutant mediates transcription of the reporter gene leading to a detectable signal, whereby decrease of said detectable signal in the presence of the test substance indicates inhibition of the MAPK pathway by said test substance.

A recombinant cell, which is transformed with an expression vector coding for the activated mutant of a MAPK pathway member of the invention may further contain an endogenously or exogenously expressible interactor molecule essential for activation of said pathway.

Thus the invention also relates to a method of screening for a substance which is an inhibitor of a MAPK which comprises the steps of (a) providing recombinant cell mixture comprising a constitutively activated mutant of a MAPK pathway member and downstream interactor molecules essential for transducing a signal through said pathway; (b) contacting said recombinant cell mixture with a test substance; and (c) determining the effect of said test substance on the interaction of the activated mutant with the interactor molecules by searching the end-point indication, wherein said effect is indicative of inhibition of the MAPK pathway by said test substance. The end-point indication may be a cell phenotype caused by activation of said MAPK pathway.

According to one embodiment, the activated mutant may optionally be expressed under an inducible promoter.

In yet another embodiment, the recombinant cell is preferably a yeast cell, and the end-point indication may be the ability of the cells to proliferate, to survive under high osmotic conditions, to mate, to invade agar and to form pseudohyphae, to sporulate or any combinations thereof.

The recombinant cell may also be a plant cell, insect cell, an avian cell or a mammalian cell, and the end-point indication may be the ability to induce apoptosis, ability to induce oncogenic phenotype or the ability to induce differentiation.

The test substance to be screened by the various embodiments of the substance-screening method of the invention may be selected from the group consisting of: protein based, carbohydrates based, lipid based, nucleic acid based, natural organic based, synthetically derived organic based, antibody based, inorganic based and peptidomimetics based substances.

More specifically, said protein based substance may be a product of any one of positional scanning of peptide libraries, libraries of cyclic peptidomimetics, peptide combinatorial library and phage display random or dedicated library.

The test substance is preferably an inhibitor of said MAPK pathway.

The invention further relates to a method of preparing a therapeutic composition for the inhibition of a MAPK pathway in a mammalian subject in need of such treatment, which method comprises the steps of:

(a) identifying an inhibitor of a MAPK pathway; and

(b) admixing said inhibitor substance with at least one of a pharmaceutically acceptable carrier, diluent, excipient and/or additive.

In a particular embodiment, the inhibitor substance is identified by the screening method of the invention.

This therapeutic composition is intended for the treatment of a pathological disorder selected from the group consisting of: neoplasia, cancer, inflammation, degenerative diseases and immunological disorders, such as arthritis for example.

Disclosed and described, it is to be understood that this invention is not limited to the particular examples, process steps, and materials disclosed herein as such process steps and materials may vary somewhat. It is also to be understood that the terminology used herein is used for the purpose of describing particular embodiments only and not intended to be limiting since the scope of the present invention will be limited only by the appended claims and equivalents thereof.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

It must be noted that, as used in this specification and the appended claims, the singular forms “a”, “an” and “the” include plural referents unless the content clearly-dictates otherwise.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

Experimental Procedures

A number of methods of the art of molecular biology are not detailed herein, as they are well known to the person of skill in the art. Such methods include site-directed mutagenesis, expression of cDNAs, analysis of recombinant proteins or peptides, Northern and Western blots analysis, transformation of yeast cells, transfection of mammalian cells, and the like. Textbooks describing such methods are e.g., Sambrook et al., Molecular Cloning A Laboratory Manual, Cold Spring Harbor Laboratory; ISBN: 0879693096, 1989, Current Protocols in Molecular Biology, by F. M. Ausubel, ISBN: 047150338X, John Wiley & Sons, Inc. 1988, and Short Protocols in Molecular Biology, by F. M. Ausubel et al. (eds.) 3^{rd} ed. John Wiley & Sons; ISBN: 0471137812, 1995. These publications are incorporated herein in their entirety by reference. Furthermore, a number of immunological techniques are not in each instance described herein in detail, as they are well known to the person of skill in the art. See e.g., Current Protocols in Immunology, Coligan et al. (eds), John Wiley & Sons. Inc., New York, N.Y.

Yeast Strains and Media

The S. cerevisiae strains used in this study were:

The pbs2Δ strain MAY1 (MATa, ura3-52, lys2-801amber, ade2-101ochre, leu2Δ1, his3-Δ200, pbs2:LEU2).

The hog1Δ strain JBY13 (MATa, leu2, ura3, his3, trp1, ade2, lys2, hog1:TRP1). The hog1Δpbs2Δ strain (JBY13 in which the PBS2 gene was disrupted).

The JBY13 and MAY1 strains were obtained from M. Gustin, Rice University [Brewster and Gustin (1994)].

The hog1Δpbs2Δ strain was produced by insertion of a pbs2:LEU2 construct into JBY13 strain. Insertion of LEU2 into the genomic PBS2 locus was verified by PCR.

Cultures were maintained on YPD (1% yeast extract, 2% Bacto Peptone, 2% glucose) or on the synthetic medium YNB-URA (0.17% yeast nitrogen base without amino acids and NH₄(SO₄)₂, 0.5% ammonium sulfate, 2% glucose, and 40 mg/l of adenine, histidine, tryptophan, lysine, leucine, and methionine).

Induction of Osmotic Shock

Ability of cells to grow under osmotic shock was tested on YPD plates supplemented with NaCl (particular salt concentrations are described for each experiment). To induce osmotic shock in liquid medium, cultures were grown to logarithmic phase (O.D600 of 0.5-1.0) at 30° C. Cells were split in half, collected by centrifugation, resuspended in the same medium or in medium containing 1M NaCl, and were collected 10 minutes later.

Mutagenesis Procedures

Library of HOG1 mutants was produced in bacterial strain LE30 according to Silhavy et al. [Silhavy (1984)]. Briefly, a plasmid carrying the HOG1 and the URA3 genes (pRS426-HOG1 obtained from Gustin; Rice University, Texas) was introduced into LE30 cells. All colonies obtained in this transformation were collected in a pool and further grown in LB medium for 24 hours during which the culture was diluted twice.

Screening of HOG1 Mutants Library

Transformation of the library of HOG1 mutants into yeast was performed as described by Schiestl and Gietz [Schiestl and Gietz (1989)]. Transformed cells were plated on selective YNB-URA plates. Colonies that grew (about 10,000 per 100 mm plate) were replica plated onto YPD plates containing different concentrations of NaCl (0.9M, 1.1M and 1.3M).

Plasmids Construction

Construction of Plasmids comprising the Different Hog1 Mutants—Constitutive Promoter:

Site-directed mutagenesis was performed by PCR or with the quick-change kit (Stratagene) according to manufacturer recommendations.

PCR/mutagenesis reaction was performed on the HOG1 gene subcloned into BlueScript SK+. Following verification of mutation, the gene was subcloned to the relevant expression vector. The sequences of the primers used for mutagenesis are listed in Table 1 and in the sequence listing.

HOG1 gene (wild type or harboring activating mutations) was introduced as a Hind3-Not1 fragment into the expression vector pES86+. PES86+ vector, containing the ADH1 promoter and the URA3 selectable, marker, was constructed by insertion of the BamH1 fragment of pADNS [Colicelli et al. (1989)] into pRS426 [Christianson et al. (1992)].

TABLE 1

Sequences of primers used in site-directed
Mutagenesis reactions

| | |
|---|---|
| | Seq |
| Primers for site-directed mutagenesis | ID |

| | | |
|---|---|---|
| Primer name | Primer sequence | No. |

| | | |
|---|---|---|
| HOG1-5′-Y68H | 5′-tcc act gca gtg ctg gcc aaa agg aca cat ctg gaa c-3′ | 2 |
| HOG1-3′-Y68H | 5′-cca aat gtc gac ctc gac gtc a-3′ | 3 |
| HOG1-5′-D170A | 5′-cgg tct agc aag aat tca agc ccc tca aat g-3′ | 4 |
| HOG1-5′-A314T | 5′-cca acg gat gaa cca gta acc gat gcc aag ttc g-3′ | 5 |
| HOG1-3′-A314T | 5′-cga act tgg cat gcc tta ctg gtt cat ccg ttg g-3′ | 6 |
| HOG1-5′-F318L | 5′-gaa cca gta gcc gat gcc aag ctc gat tgg cac-3′ | 7 |
| HOG1-3′-F318L | 5′-gtg cca atc gag ctt ggc atc ggc tac tgg ttc-3′ | 8 |
| HOG1-5′-F318S | 5′-gaa cca gta gcc gat gcc aag tcc gat tgg cac-3′ | 9 |
| HOG1-3′-F318S | 5′-gtg cca atc gga ctt ggc atc ggc tac tgg ttc-3′ | 10 |
| HOG1-5′-W320R | 5′-cga tgc caa gtt cga tcg gca ctt taa tga cgc-3′ | 11 |
| HOG1-3′-W320R | 5′-gcg tca tta aag tgc cga tcg aac ttg gca tcg-3′ | 12 |
| HOG1-5′-F322L | 5′-gcc aag ttc gat tgg cac ctt aat gac gct gat c-3′ | 13 |
| HOG1-3′-F322L | 5′-gat cag cgt cat taa ggt gcc aat cga act tgg c-3′ | 14 |
| HOG1-5′-W332R | 5′-gat ctg cct gtc gat acc cgg cgt gtt atg atg tac-3′ | 15 |
| HOG1-3′-W332R | 5′-gta cat cat aac acg ccg ggt atc gac agg cag atc-3′ | 16 |
| NP-1(T174A; | 5′-caa gac cct caa atg gca ggc ttt gtt tcc act aga tac tac agg g-3′ | 17 |
| Y176F) | | |
| NP-2(T174A; | 5′-ccc tgt agt atc tag tgg aaa caa agc ctg cca ttt gag ggt ctt g-3′ | 18 |
| Y176F) | | |
| QC-D170A1 | 5′-gtc tag caa gaa ttc aag ccc ctc aaa tga cag gc-3′ | 19 |
| (+Y176F) | | |
| QC-D170A2 | 5′-gcc tgt cat ttg agg ggc ttg aat tct tgc tag ac-3′ | 20 |
| (+Y176F) | | |

Construction of Met3-Hog1 Plasmids:

HOG1 genes (wild type or mutants) were subcloned as Hind3-Not1 fragments into Met425 plasmid (described below).

Construction of the Double Mutants—Using the Constitutive Vector ES86+

A. Construction of the six double mutants containing the N-terminal Y68H mutations has been performed as follows:

A HOG1 5′ primer bearing the Y68H mutation and a BamH1 3′ primer (see primer sequences of SEQ ID No. 21 and 22 below) were used to amplify HOG1 genes in a series of PCR reactions. The templates used were the various HOG1 mutants each bearing a different mutation of the six C-terminal mutations, resulting in fragments yielding both Y68H and one of the C-terminal mutations. These fragments were digested with PstI (a fragment containing both mutations) and ligated into a Bluescript SK+-HA-HOG1 plasmid digested also with PstI. Only correct orientations were selected. In turn, the relevant HA-HOG1 double mutants were digested from the SK+ plasmid with both Not1 and HindIII and ligated into the PES86+ yeast expression vector (a constitutive vector).

Primers sequences used in the PCR reaction:

| | |
|---|---|
| 5′ HA-HOG1 | tcc act gca gtg ctg gcc aaa agg aca cat cgt gaa c, |
| | is also denoted by SEQ ID No. 21. |
| BamHI 3′ HOG1 | cta gcc gga tcc tta ctg ttg gaa ctc att agc, |
| | is also denoted by SEQ ID No. 22. |

B. Construction of the Six Double Mutants containing the N-Terminal D170A Mutation:

The PES86⁺-HA-D170A HOG1 plasmid was digested with NotI and SalI resulting in a backbone bearing the vector fused to half of the HOG1 gene containing the D170A mutation. The same double digestion was applied to each of the six C-terminal mutants providing six fragments of the second half of the HOG1, each harboring a different C-terminal mutation. Each of these fragments was ligated to the D170A digested with NotI and SalI.

All twelve double mutant clones (FIG. 13A) were sequenced to ensure the existence of both point mutations and the absence of any other mutation in the entire gene.

Construction of the Double Mutants—Using the MET3 Inducible System

The twelve double mutants described above were subcloned into the MET3 system by inserting each of the doubly mutated HOG1 genes as HindIII and NotI fragments into the Met 425 plasmid, bearing the MET3 promoter digested with the same enzymes. The plasmid used is a derivative of the p425 MET25 plasmid in which the MET25 promoter was replaced with the MET3 promoter [Cherest, et al., (1984)].

Plasmid Loss Assay

Plasmid loss assay for positive colonies was performed by streaking patches of positive colonies on YPD plates, allowed them to grow for 24 hours and isolating single colonies.

These single colonies were replica plated onto YNB-URA plates as well as onto NaCl containing YPD plates.

RNA Preparation, Primer Extension Analysis and Sequences Analysis

RNA preparation, primer extension analysis and sequences of the primers were previously described [Engelberg et al. (1994); Stanhill et al. (1999)]. Briefly, for RNA preparation and analysis, cells were grown in 100 ml YNB-URA to an O.D600 of 0.5-0.9, split and collected by centrifugation. Half were induced for 90 minutes with YNB-URA containing 1M NaCl, while the other half was resuspended in YNB-URA. After induction, cells were centrifuged and frozen in liquid nitrogen.

Preparation of Cell Lysates and Western Blot Analysis

50 ml cell cultures were grown to an O.D600=0.5-1.0. Half of the culture was induced with 1M NaCl for 10 minutes, as described above. Cultures were pelleted and re-suspended in 8 ml DDW (double distilled water) and 10 ml TCA 20%. After repelleting the samples, they were resuspended in 200 μl TCA 20% at room temperature and 650 mg glass beads were added. 2×4′ vortex was applied on each sample. Supernatants were transferred to new eppendorf tubes and glass beads were rinsed twice with 200 μl TCA 5% (the final concentration of TCA is 10%).

Following centrifugation, pellets were resuspended in 200 μl of 2×Laemmli sample buffer followed by addition of 100 μl of 1M Tris base. Samples were vortexed for 30 second and boiled for 3 minutes prior to centrifugation, supernatant was used. SDS-PAGE, Western blot and ECL reaction for identification of the HA-Hog1 protein and the phosphorylated Hog1 were performed as described in Sambrook et al. [Sambrook (1989)].

Antibodies

The antibody for the HA tag, a monoclonal antibody designated 12CA5, was purchased from ROCHE. This antibody was used in dilution of 1/1000.

For identification of the double phosphorylated Hog1, α-P-p38 antibodies (New England Biolab) were used (diluted 1/2,500).

The secondary antibodies, anti-mouse and anti-rat, respectively, were purchased from JACKSON and were diluted 1/10,000.

Preparation of Native Cell Lysates and In Vitro Kinase Assay

200 ml cell cultures were grown to an O.D600 of 0.5-1.0. After a 10-minute induction with 1M NaCl, as described above, cells were centrifuged at 4° C. and the pellet was washed with 50 ml ice cold DDW. Following centrifugation the pellet was resuspended in 1-2 volumes of ice cold lysis buffer (50 mM Tris-HCl pH-7.4, 0.25M NaCl, 0.1% NP-40, 5 mM EDTA, 1 mM PMSF, 10 μg/ml Leupeptin, 10 μg/ml Trypsin inhibitor, 10>g/ml Pepstatin A, 313 μg/ml Benzamidine, 1 mM Na-Vanadate, 10 mM NaF, 1 mM pNPP, 10 mM α-glycerol-P). 600 mg glass beads were added and 8×1′ vortex was applied. Samples were centrifuged at 800×g for 5 minutes and supernatant was centrifuged again at 15,000×g for 15 minutes at 4° C. Sarnples were aliquoted to small volumes (˜200%1) and frozen immediately in liquid nitrogen.

For immunoprecipitation of the Hog1 protein, 10 μl of 50% Protein A were washed with ice cold PBS buffer and preconjugated with 5 μl of the anti-HA-tag 12CA5 antibody over night at 4° C. Following two washes with PBS buffer and then one wash with lysis buffer, the beads were resuspended in lysis buffer and 300 μg of the lysates (obtained as described above) were added. Samples were incubated for 1 hour at 4° C. Then, three washes with lysis buffer followed by three washes with kinase buffer (25 mM HEPES pH-7.5, 20 mM MgCl2, 20 mM α-glycerol-P, 10 mM pNPP, 0.5 mM Na Vanadate, 1 mM DTT) were performed. The kinase reaction was performed by re-suspending the beads in 10 μl kinase buffer containing 20 μM ATP, 5 μCi [α³²P] ATP and 9 μg GST-ATF2 as a substrate. GST-ATF2 construct was obtained from H. Itoh (Tokyo Institute of Technology). The reaction took place at 30° C. for 30 minutes and was stopped by transferring the samples to ice and adding 10 μl Laemmli loading buffer×4 prior to boiling.

Kinase reactions were loaded on SDS-PAGE, which was run, dried under vacuum and exposed to x-ray film.

Expression, Purification and Analysis of Human p38α

Wild type and active mutants of p38α were expressed in E. coli using the pET15b expression vector (Novagen) so that all proteins contained a polyhistidine tag in their NH2-terminus. p38 cDNA (wild type or harboring activating mutations) was inserted into pET15b vectors as a Sap1-NruI fragment following treatment with klenow fragment of DNA poll. Growth conditions, induction of expression and protein purification were performed according to Wang et al. [Wang et al. (1997)]. Purified proteins were assayed as previously described [Jiang et al. (1997)] using GST-ATF2 (20 μg) as a substrate.

### Example 1

Isolation of Point Mutations in HOG1 which Render it Independent of Pbs2 Activation

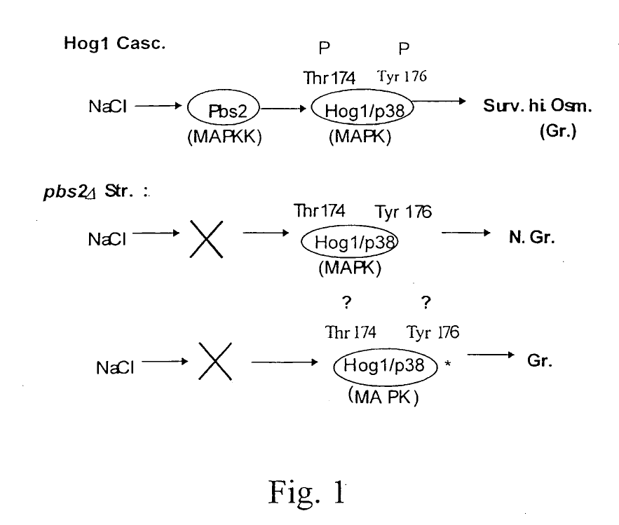

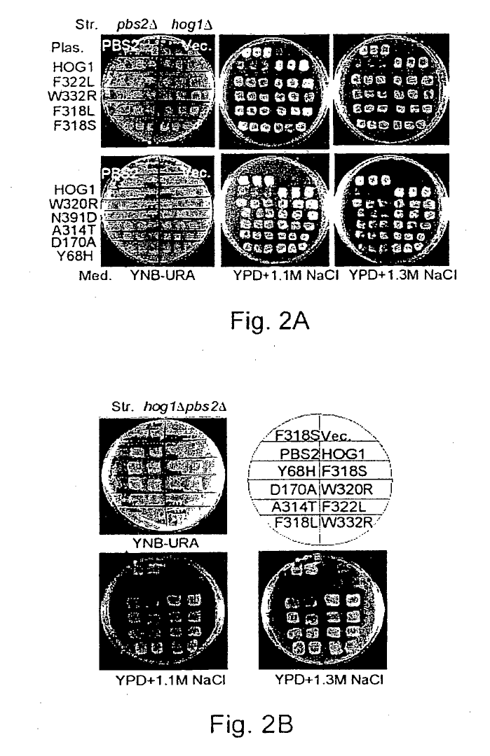

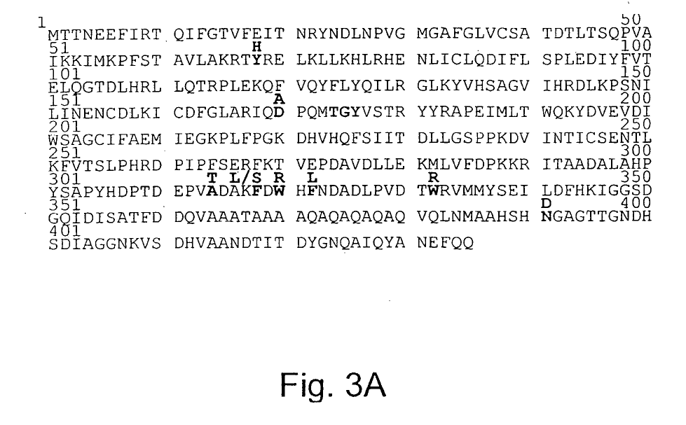

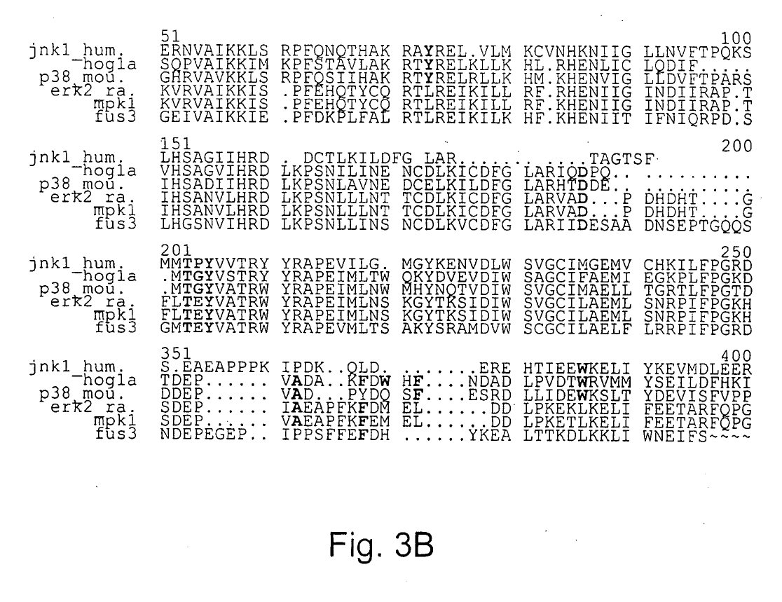

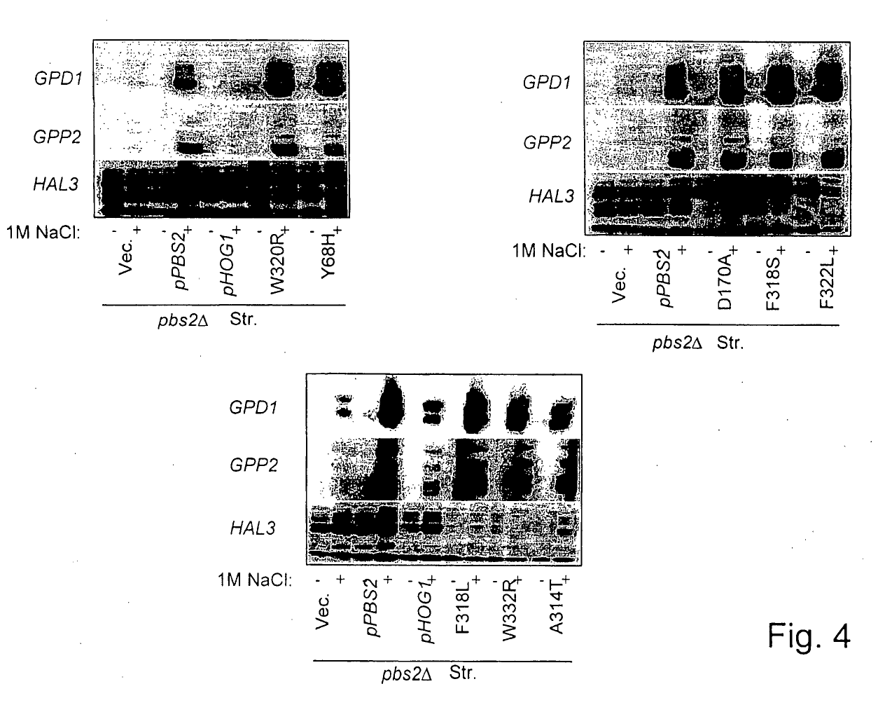

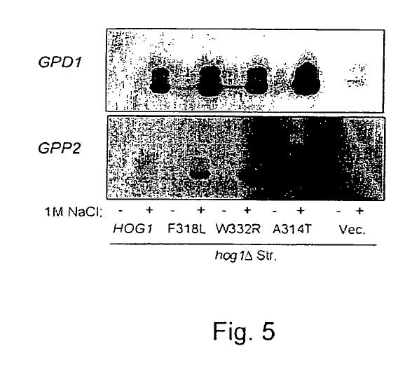

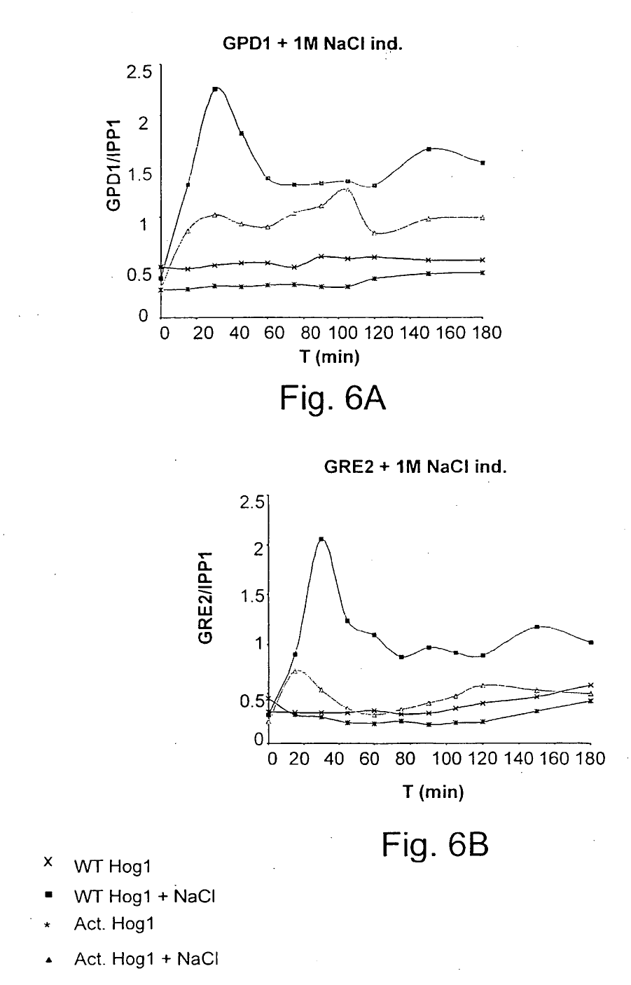

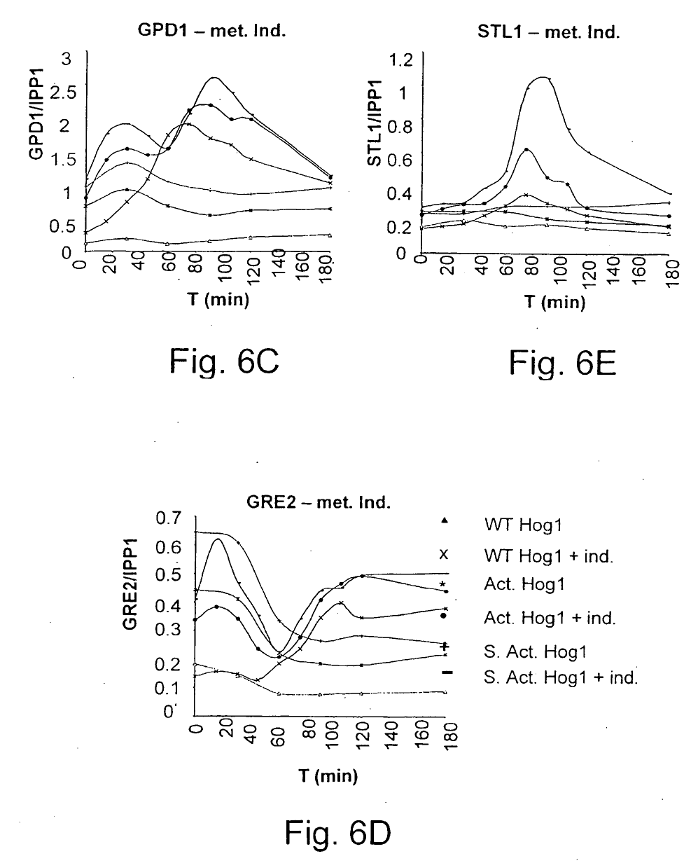

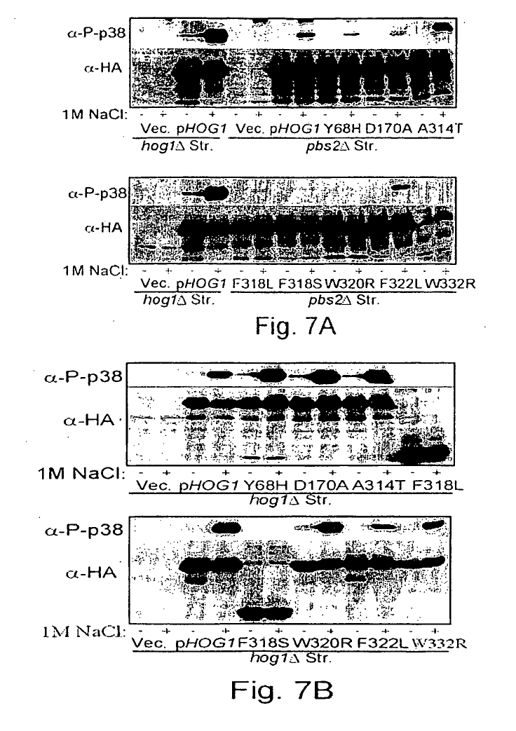

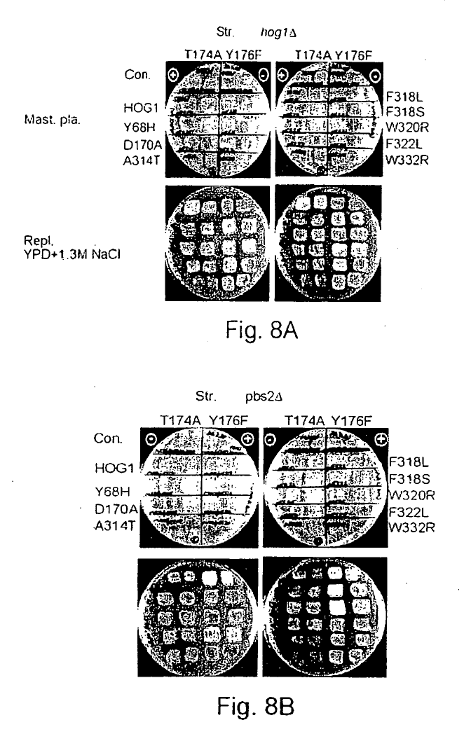

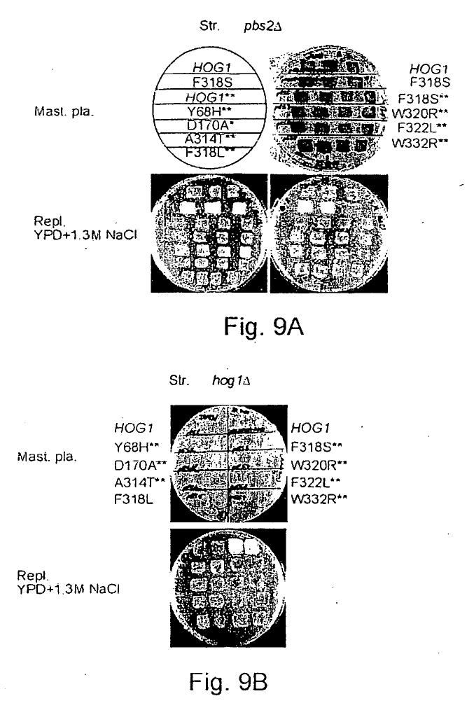

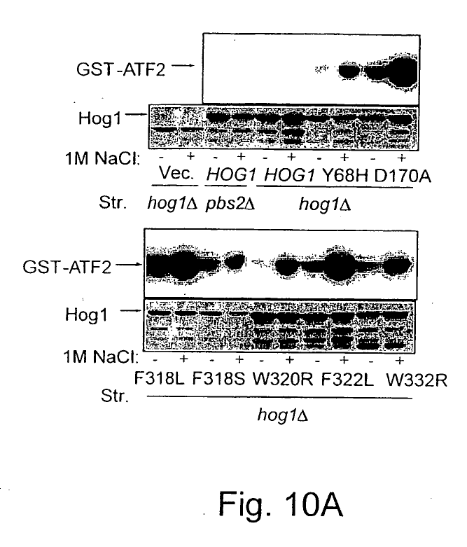

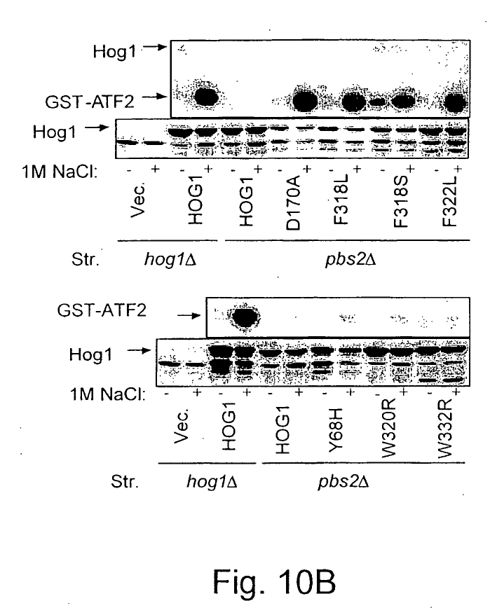

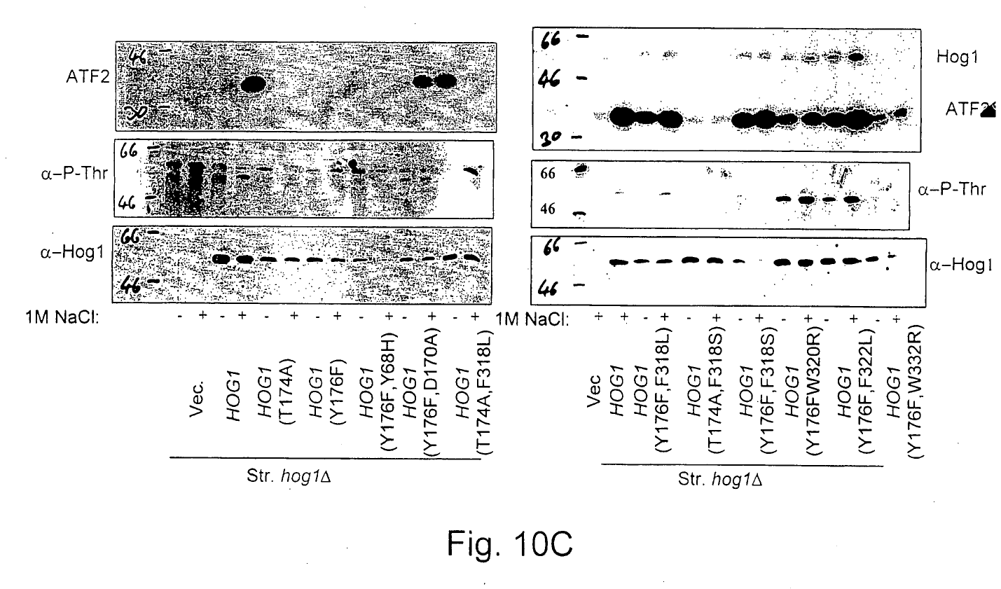

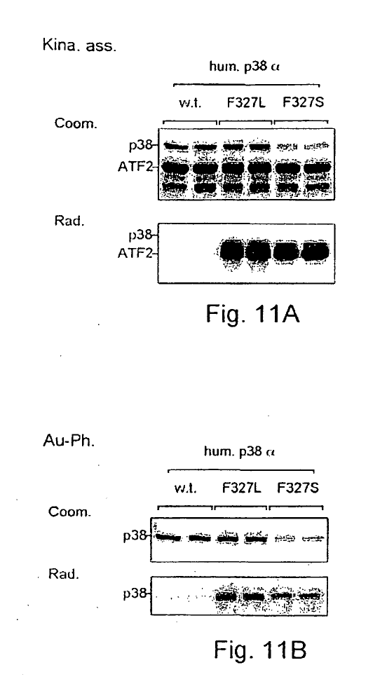

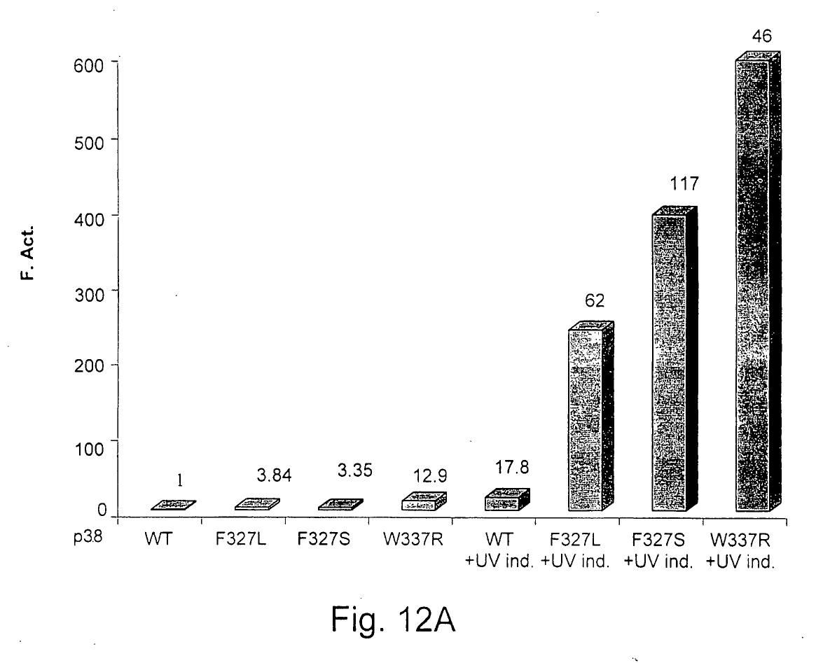

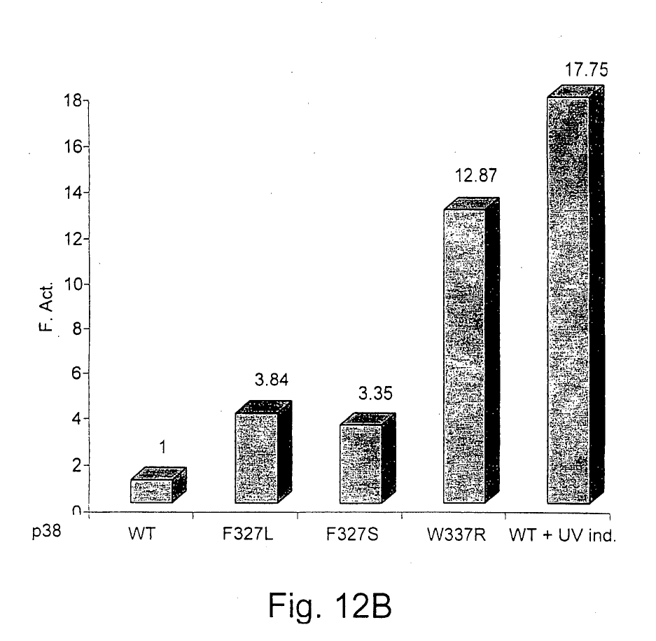

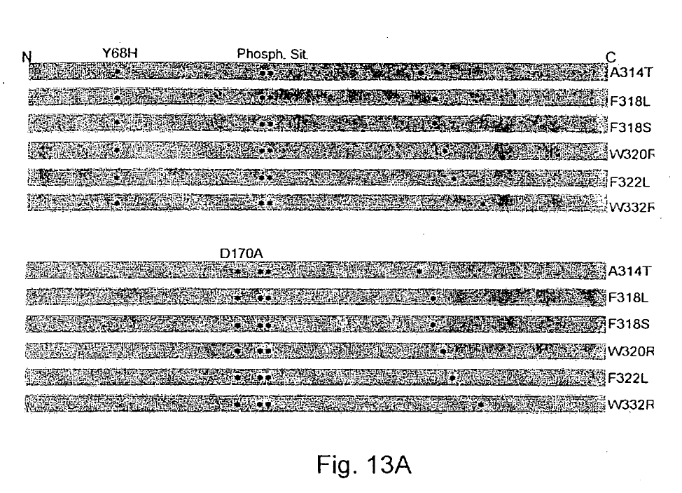

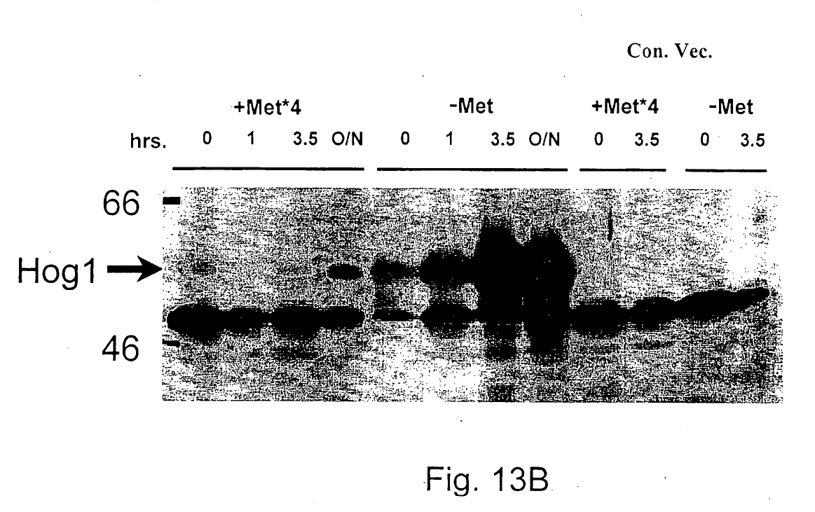

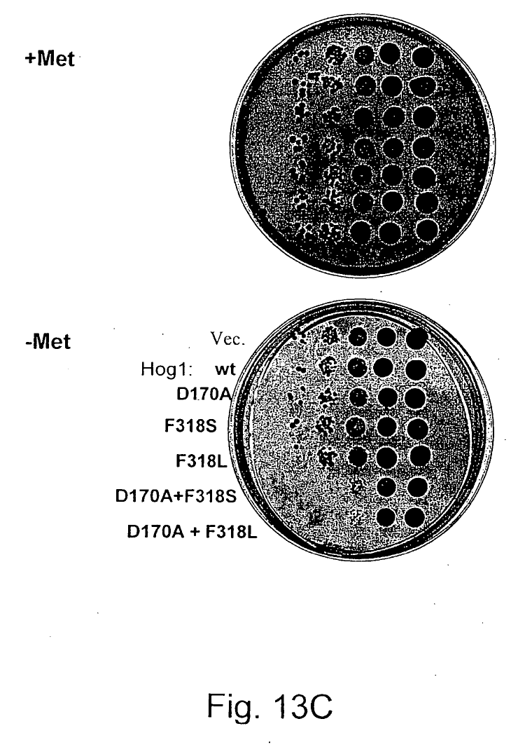

In screening for active forms of MAPKs, the inventors took advantage of the pbs2Δ strain, which lacks the MAPKK Pbs2 and therefore, cannot grow under high osmotic conditions. Overexpression of the MAPK Hog1 (which is the Pbs2 substrate) does not enable pbs2Δ cells to grow on salt (FIG. 2A; second row in each plate), demonstrating the absolute dependency of Hog1 activity on Pbs2-mediated phosphorylation. This result forms the basis for the inventors premise that only a constitutively active form of Hog1 might enable pbs2Δ cells to grow on salt. The idea is therefore to produce a library of plasmids of HOG1 mutants, with a view to find one or a few mutants that have gained intrinsic catalytic activity, which is independent of Pbs2 activation. The rare active mutants will be identified by introducing the library to pbs2Δ cells and selecting colonies on salt. Each colony that grows should harbor a constituvely active, Pbs2 independent Hog1 kinase (see FIG. 1).

Therefore, a library of HOG1 mutants was produced [Silhavy T J (1984); Michaeli et al. (1989)] and introduced into a pbs2Δ strain [Brewster et al. (1993)]. About 5×10⁵ transformants were obtained and allowed to grow on selective media (Y-NB with no uracil) in the absence of salt. Then colonies were replica-plated to plates supplemented with NaCl (0.9M-1.3M), and about 150 positive colonies (colonies that grew on salt concentration of 1.1 M NaCl or higher) were collected. The linkage between growth on salt and the library plasmid was verified through a plasmid-loss assay and then by re-transfection (following purification from yeast) to pbs2Δ cells. Clones which have passed these tests (41-clones, data not shown), were considered as true positives, as summarized by Table 2A. FIG. 2A shows that the isolated Hog1 mutants enable pbs2Δ cells to grow in the presence of salt, whereas the wild type Hog1 could not support any growth of expressing cells.

The 41 true positive clones were next sequenced and each clone was found to contain a single point mutation in the coding sequence of Hog1 (which is also denoted by SEQ ID No. 1). Many of the clones harbor an identical mutation (Table 2B) suggesting that the screen was saturated. As indicated by Table 2B; and FIGS. 2A and 3, nine different point mutations were identified. To show unequivocally that each of these point mutations is sufficient to render Hog1 independent of Pbs2, each mutation was introduced to a wild type HOG1 gene through site directed mutagenesis, and the resulting mutants were expressed in pbs2Δ cells. All nine mutations allowed growth in the presence of salt, indicating that indeed each point mutation is sufficient to render Hog1 independent of Pbs2.

The results shown so far demonstrate that the HOG1 variants isolated in the screen are independent of their MAPKK, Pbs2. As MAPKs are probably active as dimers [Cobb and Goldsmith 2000; Khokhlatchev et al. (1998)], the possibility remains that the mutants are not exclusively independent, but dimerize with the endogenous Hog1 that is expressed in the pbs2Δ strain. The ability of the active mutants to allow pbs2Δhog1Δ double knockout strain to grow on salt was therefore next tested. As expected, this strain is not rescued by overexpression of either Hog1, or Pbs2 (FIG. 2B). Yet, it is rescued by the Hog1 mutants (FIG. 2B), indicating that these mutants are biologically active, and functioning alone, independently of Pbs2 and endogenous Hog1.

TABLE 2
Screen Results

| A | | |
|---|---|---|
| | # of colonies | % of transformants |
| Total colonies | 5 × 10⁵ | 100 |
| obtained | | |
| Total positives | 150 | 0.03 |
| True positives | 41 | 0.0082 |

| B | |
|---|---|
| | # of colonies harboring |
| Mutation identity | the mutation |
| Y68H | 4 |
| D170A | 1 |
| A314T* | 1 |
| F318L | 17 |
| F318S | 3 |
| W320R | 2 |
| F322L | 3 |
| W332R | 9 |
| N391D* | 1 |

*For unknown reasons, two mutants, N391D and A314T, were not able to rescue pbs2Δ cells when expressed from a strong promoter and containing a HA-tag. Therefore these mutants were studied only in some of the further experiments.

Six of the Activating Mutations are Located in the Dimerization Domain

FIG. 3A shows the location of the mutations within the HOG1 linear sequence. Strikingly, six of the nine mutations are located in a short stretch of residues, between amino acids 314 and 332 (as located within the amino acid sequence of the HOG1 protein substantially as denoted by SEQ ID No. 1), which forms part of the L16 domain. These mutated residues appear to be in close proximity to (but not within) the docking domain suggested recently by Nishida's group [Tanoue et al. (2000)].

The other two mutations are located at the N terminus. One mutation (D170A) is located just 4 amino acids from the phosphoacceptor Thr174. The second mutation in the N-terminus is Y68H. Alignment of the HOG1 sequence with the sequences of mammalian MAPKs as shown by FIG. 3B, reveals that all mutations but one, W320R, occur in residues which are conserved in at least one subfamily.

The amino acid sequences of the different mammalian MAPKs, human JNK, mouse p38, rat ERK and the yeast mpk1 and fus3, as disclosed by FIG. 3B are based on the amino acid sequence as provided by the GeneBank accession numbers: P45983, p47811, M64300, Q00772 and P16892 respectively, which are incorporated herein by reference.

### Example 2

Activating Mutations Allow pbs2Δ Cells to Grow on High Salt by Activating the Authentic Hog1 Cascade

The Pbs2/Hog1 cascade allows growth under osmotic stress through induction of transcription of genes which encode enzymes involved in glycerol biosynthesis [Albertyn et al. (1994); Akhtar et al. (1997); Gustin et al. (1998)]. Therefore, the next step was to verify that the active Hog1 mutants allow pbs2Δ cells to grow on salt by activating the authentic pathway downstream to Hog1, namely, induction of GPD1 and GPP2 gene expression. Thus, RNA was prepared from pbs2Δ cells harboring various plasmids (vector, HOG1 and HOG1 mutants) that were either exposed, or not exposed to 1.0M NaCl, and the levels of GPD1 and GPP2 mRNAs were then measured. GPD1 encodes glycerol-3-phosphate dehydrogenase and GPP2 encodes glycerol-3-phosphatase, two enzymes involved in glycerol biosynthesis whose transcription is HOG1 dependent [Akhtar et al. (1997); Gustin et al. (1998)].

As shown in FIG. 4, pbs2Δ cells over-expressing wild type HOG1 are not capable of inducing high levels of GPP2 or GPD1 mRNA following exposure to NaCl. On the other hand pbs2Δ cells expressing the HOG1 mutants induced transcription of wild type levels of GPD1 and GPP2 mRNA. Furthermore, most of the active Hog1 molecules induced an increase in GPD1 transcription in cells grown under non-induced conditions.

Thus, the HOG1 mutants isolated in the present invention's screen are capable of activating the entire authentic pathway downstream to Hog1, independent of Pbs2.

The effects of Hog1 mutants on GPP2 and GPD1 transcription in hog1Δ cells (FIG. 5) were tested as well. This analysis showed that the mutants tested induced high levels of GPP2 and GPD1 mRNAs, significantly above the levels induced by wild type Hog1 (FIG. 5). Note that these Hog1 mutants induced GPD1 transcription in the absence of salt suggesting that they may be constitutively active. The level of GPD1 induced under these conditions is however far below maximum, an expected result given that Hog1 activation is required, but not sufficient to induce maximal transcription rate from this promoter [Marquez et al. (1998)]. The minor effect is also expected as high expression of these genes result in growth arrest. Therefore a feedback inhibition response led to lower transcription of some Hog1 target genes. However, when an inducible expression system was used to express the active Hogs mutants (as indicated below, FIGS. 6C-6D) a full activation of target genes was obtained short time after induction, verifying that the mutants fully activate the pathway downstream to Hog1.

Inducible Expression of Hog1 Mutants

To further demonstrate that the Hog1 mutants are directly reponsible for induction of specific gene expression the active Hog1 molecules have been expressed using the inducible Met3 promoter [Cherest, (1984)]. Expression in this inducible system can be obtained upon removal of methionine from the growth medium. As shown in FIGS. 6C-6E, induction of expression of the active Hog1 molecules by removal of methionine (without any other treatment—e.g., osmotic shock) was sufficient to induce transcription of the Hog1 dependent genes STL1, GPD1 and GRE2. Induction of expression of wild type Hog1 protein did not give rise to significant transcription of these genes. Induction of Hog1 expression upon removal of methionine was verified by Western blot (data not shown).

### Example 3

Most of the Active Hog1 Mutants are not Phosphorylated on Thr174 and Tyr176 in pbs2Δ Cells

The results shown above suggest that the Hog1 mutants perform all Hog1 molecular and biological functions in cells lacking Pbs2. As a trivial explanation of what makes the Hog1 molecule independent of the Pbs2 molecule, could be that the Hog1 mutants acquired an affinity to another MAPKK, which phosphorylates and activates them. As at least five MAPK cascades function in yeast [Gustin et al. (1998); Madhani and Fink (1998)], there should be at least four intact MAPKKs in the pbs2Δ cells. To address this possibility, the phosphorylation status of the Hog1 mutants were measured by Western blot analysis. An anti-phospho-p38 antibody that recognizes dually phosphorylated Hog1 [Reiser et al. (1999)] was used for that purpose. Whole cell extracts were prepared from pbs2Δ and hog1Δ cells expressing wild type Hog1, or the various mutants. Extracts were prepared from cells that were either exposed, or not exposed to salt (1M NaCl). The Western blot analysis revealed several phosphorylation patterns as demonstrated by FIG. 7. When expressed in pbs2Δ cells, the Hog1 mutants F318S, W320R and W332R showed no detectable phosphorylation (FIG. 7A). Hog1 mutants Y68H, D170A and F318L show low levels of phosphorylation in pbs2Δ cells. This level of phosphorylation is below the level measured in wild type Hog1 expressed in pbs2Δ cells. As can be seen in FIG. 7A lane 8 in the upper panel, wild type Hog1 is phosphorylated (to a low level relative to its phosphorylation in hog1Δ cells) in pbs2Δ cells exposed to osmotic stress. The kinase responsible for this low level of phosphorylation remains unidentified. A possible explanation could be autophosphorylation activity [Brill et al. (1994); Jiang et al. (1997)]. It should be stressed that the low phosphorylation level also observed in unmutated Hog1 in pbs2Δ cells was insufficient to allow growth on salt (FIG. 2A). It is therefore likely that the low level of phosphorylation measured in Y68H, D170A and F318L is not responsible for their biological activity in pbs2Δ cells. This notion has been addressed by mutagenizing the phosphoacceptors Thr174 and Tyr176 in all the mutants (see below). However, Hog1 mutants A314T and F322L expressed in pbs2Δ cells seem to be phosphorylated at equivalent levels to that of wild type Hog1 in these cells (FIG. 2A).

Hog1 mutants show a different pattern of phosphorylation when expressed in hog1Δ cells (FIG. 7B). First, mutants Y68H, D170A, A314T and W320R are phosphorylated even in the absence of salt stimulation, at a significantly higher level than that of the wild type Hog1. Second, the level of their phosphorylation following induction with salt is equivalent and even higher than that of wild type. Thus, the presence of Pbs2 is important for increased phosphorylation of these mutants (compare FIGS. 7A to 7B), although the presence of Pbs2 is not required for their biological activity (e.g. growth, as demonstrated by FIG. 2). Strikingly, mutants F318L and F318S expressed in hog1Δ seem to be labile. As shown in FIG. 7B (lanes 11, 12 of the upper panel and lanes 5, 6 of the lower panel), in lysates prepared from hog1Δ cells expressing Hog1^{F318L} or Hog1^{F318S}, a 35 kDa protein reacted with the anti-HA antibodies. Normally, the apparent molecular weight of native Hog1 is about 50 kDa. Note that Hog1^{F318L} and Hog1^{F318S} molecules are fully functional in hog1Δ cells and allow growth on salt (FIG. 2).

In summary, the Western blot analysis suggests that in pbs2Δ cells, most mutants are not activated through dual phosphorylation and are probably independent of any upstream MAPKK activity. This conclusion is also strongly supported by the results obtained using the active mutants, which were also mutated in their phosphoacceptor residue Tyr176. (see example 4 below). Mutations F318L and F318S probably cause dramatic structural changes, which expose the kinase to proteolytic activity.

### Example 4

Properties of the Active Hog1 Variants Mutated at the Double Phosphorylation Motif

To further investigate the role of phosphorylation, the phosphoacceptors Thr174 and Tyr176 of each of the mutants have been mutated. It was expected that all mutants (and in particular F318S, W320R and W332R, which were not phosphorylated at all in pbs2Δ cells), would tolerate replacement of Thr174 with Ala and Tyr176 with Phe and would remain active. As can be seen in FIG. 8, replacement of Tyr176 with Phe had no effect on the ability of all the mutants to allow growth of both hog1Δ cells and pbs2Δ cells (see also FIG. 9 for Hog1^{D170AY176F}) suggesting that phosphorylation of Tyr176 is dispensable for their activity. This result confirms that all the mutants are independent of upstream mediated dual phosphorylation. Unexpectedly, when Thr174 residues were mutated to Ala the mutants lost their ability to rescue either hog1Δ or pbs2Δ cells (FIG. 8). Similarly, when both T174 and Y176 were changed to Ala and Phe respectively, the mutants could not support growth on salt (FIG. 9). The finding that most Hog1 mutants do not require dual phosphorylation for their activity (FIG. 7A), but on the other hand mutating Thr174 abolishes their activity is intriguing. It is possible that phosphorylation of Thr174 is not required, but replacement of Thr174 altogether inactivates the catalytic core of the enzyme. Support for this notion comes from Robbins et al., who showed that replacing the Thr183 phosphoacceptor in ERK2 with Glu, not only prevented increasing activity, but in fact dramatically reduced the maximal kinase activity [Robbins et al. (1993)]. An alternative possibility is that the Hog1 mutants acquired autophosphorylation activity that selectively phosphorylates Thr174.

### Example 5

The mutated Hog1 Variants are Catalytically Active in the Absence of Salt Induction

The fact that the active Hog1 variants activate the authentic Hog1 pathway independently of upstream phosphorylation, suggests that they may have acquired intrinsic catalytic activity. This notion is also supported by the location of the mutated residues, as demonstrated by FIG. 3, and by the induction of salt independent target genes transcription (FIGS. 4 to 6). Therefore, the inventors next tried to reveal the biochemical basis of their unusual properties. The most attractive explanation would be that these mutated molecules have acquired intrinsic catalytic activity. To directly test whether the mutants are catalytically active, conditions for an in vitro kinase assay for Hog1 were established. Each of the mutant Hog1 molecules as well as the wild type molecule was expressed in hog1Δ or in pbs2Δ cells, immunoprecipitated from these cells and assayed in vitro for its ability to phosphorylated ATF2 as demonstrated by FIG. 10. The catalytic activity of all the mutants was found to be extremely high when expressed in hog1Δ cells (FIG. 10A). Interestingly, most mutants exhibited very high catalytic activity in the absence of any stimulation. Under these conditions, the activity of wild type Hog1 molecules is barely detectable (FIG. 10A upper panel, lane 3). Particularly high basal kinase activity was measured for Hog1^{F318L} whereas Hog1^{D170A}, Hog1^{F318S}, Hog1^{F322L} and Hog1^{W332R} exhibited somewhat lower basal activity. Hog1Y68H and Hog1^{W320R} showed relatively low catalytic activity, yet much higher than that of wild type Hog1. The activity of most mutants further increased when salt was added to the culture. However, only minor increase was observed for Hog1^{F318L} and no increase was measured for Hog1^{F318S} in the presence of salt (FIG. 10). Thus, Hog1^{F318L} and Hog1^{F318S} manifest their maximal catalytic activity under any growth conditions and could be regarded as constitutively active molecules. Most mutants were also catalytically active in pbs2Δ cells although to a lower level (FIG. 10B). While the wild type Hog1 kinase showed no catalytic activity in these cells, mutants Hog1^{D170A}, Hog1^{F318L}, Hog1^{F318S} and Hog1^{F322L} were found to be highly active.

Furthermore, these mutants exhibited a significant level of basal catalytic activity, suggesting that they are totally independent of Pbs2 and of salt induction. Unlike mutants Hog1^{D170A}, Hog1^{F318L}, Hog1^{F318S} and Hog1^{F322L}, mutants Hog1^{Y68H}, Hog1^{W320R} and Hog1^{W332R} did not show a significant increase in catalytic activity (FIG. 10B, lower image). Only a slight increase in activity was measured following salt induction in Hog1^{Y68H} and Hog1^{W320R} (FIG. 10B).

These findings pose few questions, how could the mutants with low kinase activity support growth on salt of pbs2Δ cells? What mechanism could be responsible for increase in activity of some mutants when salt is provided? This latter question is particularly interesting in pbs2Δ cells where the mutants are not phosphorylated. It could be that activation of Hog1 requires the removal of an inhibitory component in addition to MAPKK dependent phosphorylation. In such a case, the salt dependent increase in the activity of the mutants is a result of this molecular event. This possibility may be supported by studies that identified several inhibitors of JNK1 including JIP [Dickens et al. (1997)] and GST [Adler et al. (1999)]. It could be that the mechanism of action of some active mutants, primarily those whose catalytic activity in pbs2Δ cells is barely measurable (Hog1^{Y68H}, Hog1^{W320R} and Hog1^{W332R}), involves their release from an inhibitory complex. Namely, their ability to rescue pbs2Δ cells stems from an increase in their catalytic activity in combination with their dissociation from inhibitory regulation. Being released from the inhibitory complex, these mutants reside in close proximity to their substrates and are able to execute all Hog1 activities.

In any case, the results shown in FIGS. 10A and 10B strongly suggest that all mutants have gained an intrinsic catalytic activity, that is independent of external stimulation. The in vitro kinase assay also revealed some degree of autophosphorylation activity in at least one mutant (Hog1^{F318S}; FIG. 10B). This activity, that was also observed in other mutants after long exposure (data not shown), suggests that the mechanism of activation of some mutants may be due to autophosphorylation (see a similar result for human p38 in FIG. 11B).

As shown in Example 4, an intact Thr 174 was essential for the mutant ability to rescue either hog1Δ or pbs2Δ cells. In order to examine the role of phosphorylation of the phosphoacceptors Thr174 and Tyr176 of the different mutants on their catalytic activity, an in vitro kinase assay was performed as described above in different activating mutants having replaced Thr174 or Tyr176. As shown by FIG. 10C, mutating the Tyr176 to Phe or Thr174 to Ala in the wild type Hog1 totally abolished activity. Converting the Tyr176 to Phe in the activating mutants did not change activity, however, converting Thr 174 to Ala abolished the activity of both F318L and F318S mutants. However, although intact Thr174 is essential for activity of the mutants it is probably required as a structural moiety and not as a phosphoacceptor. This notion is supported by the results described below that show no Thr phosphorylation (FIG. 10C—middle panel) of many of the highly active mutants (FIG. 10C upper panel). Altogether, it seems that the mutants acquired an intrinsic activity that is independent of any-kind of external stimulation or any regulator.

In addition to their catalytic activity of the Hog1 mutants in the absence of any salt (NaCl) induction, they significantly affect biological properties of the cell. Growth rate under optimal growth conditions of cells expressing the mutants was dramatically slowed. Generation time of these cultures is about two times longer than that of cells expressing wild type Hog1. Also, the active mutants increase cell aggregation and flocculation (data not shown). These effects disclose a novel role for Hog1 in growth arrest and cell-cell interaction.

### Example 6

Insertion of Equivalent Mutations to the Human p38α Renders the Kinase Catalytically Active

As the activating mutations in HOG1 occur in residues that are conserved in many MAPKs, including those of mammals (FIG. 3B), it is possible that similar mutations would activate other MAPKs. To test this hypothesis, the Phe327 residue of the human p38α gene (homolog of Phe322 in HOG1) was replaced with Leu, or Ser. The p38^{Phe327Leu}, p38^{Phe327Ser}, as well as a wild type p38 protein, were expressed in E. coli utilizing the pET15 vector, and purified using Ni²⁺-Agarose. The recombinant purified proteins were tested for their kinase activity in vitro (FIG. 11A). As expected, wild type p38 exhibited very low catalytic activity. The mutants however, exhibited: significant kinase activity (at least 70 fold higher than wild type; FIG. 11A).

This result validates, the notion that mutations identified in the HOG1 screen could be used to produce active forms of mammalian MAPKs. As the mutated p38 enzymes are active as recombinant proteins expressed in bacteria, it is clear that they have acquired intrinsic catalytic activity, which is independent of activation by any upstream MAPKK.

Analyzing the in nitro kinase assay results of the, invention (FIG. 11A), a phosphorylated protein of 38 KDa was noticed. This phosphorylated protein appeared only when active p38 mutants were used (FIG. 11A). As this protein is almost certainly autophosphorylated p38, the autophosphorylation activity of these mutant enzymes was examined.

The inventors found that the p38^{Phe327Leu} and p38^{Phe327Ser} acquired the intrinsic capability for autophosphorylation (FIG. 11B). Thus, similar to the case of some of the Hog1 mutants, the ability to autophosphorylate most probably accounts for the absolute independence of the p38 mutants of upstream MAPKKs. However, autophosphorylation activity of wild type p38 was barely detectable as demonstrated by FIG. 11B.

In order to further analyze in a tissue culture assay, the possibility to create functional equivalent Hog1 mutants within the human p38 molecule, the three human p38 mutants F327L, F327S and W337R have been cloned in the mammalian expression vector pcDNA3. These p38 activated mutants as well as the p38 wild type genes were introduced into COS-7 cells in duplicates. Stimulation of p38 was performed by exposing one set of plates to UV radiation, forty eight hours after transfection. One hour later protein lysates were prepared, transfected p38 molecules were immunoprecipitated and assayed in a kinase assay using GST-ATF2 as a substrates. Kinase assay mixtures were loaded on a SDS-PAGE gel that was ran and dried. Incorporation of radioactive phosphates to the substrate were quantitated in a phosphoimager. To calculate the fold induction, the level of incorporation in the assay with the wild type molecule (in the cells that were not exposed to UV radiation) was set as 1. As shown by FIG. 12A, mutated p38 molecules that were exposed to UV stimulation performed significant elevation in kinase activity compared to the wild type culture. Even in the absence of UV stimulation the intrinsic catalytic activity of the three mutants was significantly higher compare to the wild type molecule (from 3.35 to 17.75 folds), as demonstrated by FIG. 12B.

### Example 7

Combination of Activating Mutations on one Hog1 Molecule Increases Dramatically the Biological Effects of the Mutants

To examine the biological effect of combining activating mutation on Hog1, six different double mutants containing the Y68H mutation and six mutants containing the D170A mutation, were prepared as described in experimental procedures and illustrated by FIG. 13A.

All double mutants, cloned into the ES86+ expression vector (a constitutive vector) were introduced into yeast strains JBY13 and MAY1. Whereas thousand of colonies appeared with most plasmids, ES86+HOG1^{D170+F318S} and ES86+HOG1^{D170A+F318L} gave no colonies, indicating that cells cannot survive expression of those double mutant Hog1 molecules. To prove this point unequivocally, all double mutants were cloned into the Met3 vector (which allows expression only when methionine is omitted from the growth medium). All resulting plasmids gave colonies. As was demonstrated in FIG. 13B, all colonies were tested for growth on plates supplemented with medium containing methionine or deprived for methionine. As can be seen in FIG. 13C, cells harboring plasmids MET3HOG1^{D170A+F318L} and MET3HOG1^{D170+F318S} were not able to grow on medium lacking methionine, suggesting that expression of the respective Hog1 proteins cause lethality.

### REFERENCES

Adler, V., Z. Yin, S. Y. Fuchs, M. Benezra, L. Rosario, K. D. Tew, M. R. Pincus, M. Sardana, C. J. Henderson, C. R. Wolf, R. J. Davis, and Z. Ronai. 1999, EMBO J. 18: 1321-1334.

Akhtar, N., A. Blomberg, and L. Adler. 1997, FEBS Lett 403: 173-180.

Albertyn, J., S. Hohmann, J. M. Thevelein, and B. A. Prior. 1994, Mol. Cell. Biol. 14: 4135-4144.

Beato, M, et al., 1987, J. Steroid Biochem, 27:9-14.

Bott, C. M., S. G. Thorneycroft, and C. J. Marshall. 1994. FEBS Lett. 352: 201-205.

Brewster, J., T. De Valoir, N. Dywer, E. Winter, and M. Gustin. 1993, Science 259: 1760-1763.

Brewster and Gustin 1994, Yeast 10, 425-439.

Brill, J. A., E. A. Elion, and G. R. Fink. 1994, Mol. Biol. Cell. 5: 297-312.

Brown, M, et al., 1987, Cell, 49:603-12.

Brunner, D., N. Oellers, J. Szabad, W. H. Biggs, 3rd, S. L. Zipursky, and E. Halen. 1994, Cell 76: 875-888.

Canagarajah, B. J., A. Khokhlatchev, M. H. Cobb, and E. J. Goldsmith. 1997, Cell 90: 859-869.

Cherest, M., et al., 1984, Gene 34:269-281.

Christianson et al. 1992, Gene 110, 119-122.

Cobb, M. H. and E. J. Goldsmith. 2000, Trends. Biochem. Sci. 25: 7-9.

Colicelli et al. 1989, PNAS 86, 3599-3603.

Cowley, S., H. Paterson, P. Kemp, and C. J. Marshall. 1994, Cell 77: 841-852.

Dickens, M., J. S. Rogers, J. Cavanagh, A. Raitano, Z. Xia, J. R. Halpern, M. E. Greenberg, C. L. Sawyers, and R. J. Davis. 1997, Science 277: 693-696.

Dong, C., D. D. Yang, M. Wysk, A. J. Whitmarsh, R. J. Davis, and R. A. Flavell. 1998, Science 282: 2092-2095.

Emrick et al. 2001, J. Biol. Chem. 276, 46469-46479.

Engelberg, D., E. Zandi, C. S. Parker, and M. Karin. 1994, Mol. Cell. Biol. 14: 4929-4937.

Galcheva-Gargova, Z., B. Derijard, I. H. Wu, and R. J. Davis. 1994, Science 265: 806-808.

Gredinger, E., A. N. Gerber, Y. Tamir, S. J. Tapscott, and E. Bengal. 1998, J Biol Chem 273: 10436-10444.

Gustin, M. C., J. Albertyn, M. Alexander, and K. Davenport. 1998, Microbiol. Mol. Biol. Rev. 62: 1264-1300.

Hall, J. P., V. Cherkasova, E. Elion, M. C. Gustin, and E. Winter. 1996, Mol. Cell. Biol. 16: 6715-6723.

Han, J., J. D. Lee, L. Bibbs, and R. J. Ulevitch. 1994, Science 265: 808-811.

Ip, Y. T. and R. J. Davis. 1998, Curr. Opin. Cell. Biol. 10: 205-219.

Jiang, Y., Z. Li, E. M. Schwarz, A. Lin, K. Guan, R. J. Ulevitch, and J. Han. 1997, J. Biol. Chem. 272: 11096-11102.

Khokhlatchev, A. V., B. Canagarajah, J. Wilsbacher, M. Robinson, M. Atkinson, E. Goldsmith, and M. H. Cobb. 1998, Cell 93: 605-615.

Kyriakis and Avruch, Physiol. Rev. 81, 801-869 (2001).

Lin, A., A. Minden, H. Martinetto, F. X. Claret, C. Lange-Carter, F. Mercurio, G. L. Johnson, and M. Karin. 1995, Science 268: 286-290.

Madhani, H. D. and G. R. Fink. 1998, Trends. Genet. 14: 151-155.

Mansour, S. J., W. T. Matten, A. S. Hermann, J. M. Candia, S. Rong, K. Fukasawa, G. F. Vande Woude, and N. G. Ahn. 1994, Science 265: 966-970.

Marquez, J. A., A. Pascual-Ahuir, M. Proft, and R. Serrano. 1998, EMBO J. 17: 2543-2553.

Marshall, C. J. 1994, Curr. Opin. Genet. Dev. 4: 82-89.

Mayo, K E, et al., 1982, Cell, 29:99-108.

Michaeli, T., J. Field, R. Ballester, K. O'Neill, and M. Wigler. 1989, EMBO J. 8: 3039-3044.

Minden, A. and M. Karin. 1997, Biochem. Biophys. Acta. 1333: F85-104.

Ono, k. and J. Han. 2000, Cellular Signalling 12: 1-13.

Pearson et al, 2001, Endocrine Rev. 22, 153-183.

Pages, G., S. Guerin, D. Grall, F. Bonino, A. Smith, F. Anjuere, P. Auberger, and J. Pouyssegur. 1999, Science 286: 1374-1377.

Pescini, R, et al., 1994, Biochem & Biophy Res Communications 202(3): 1664-7.

Raingeaud, J., S. Gupta, J. S. Rogers, M. Dickens, J. Han, R. J. Ulevitch, and R. J. Davis. 1995, J. Biol. Chem. 270: 7420-7426.

Reiser, V., H. Ruis, and G. Ammerer. 1999, Mol. Biol. Cell. 10: 1147-1161.

Robbins, D. J., E. Zhen, H. Owaki, C. A. Vanderbilt, D. Ebert, T. D.

Geppert, and M. H. Cobb. 1993, J. Biol. Chem. 268: 5097-5106.

Robinson, M. J. and M. H. Cobb. 1997, Curr. Opin. Cell. Biol. 9: 180-186.

Robinson, M. J., S. A. Stippec, E. Goldsmith, M. A. White, and M. H. Cobb. 1998, Curr. Biol. 8: 1141-1150.

Sabapathy, K., Y. Hu, T. Kallunki, M. Schreiber, J. P. David, W. Jochum, E. F. Wagner, and M. Karin. 1999, Curr. Biol. 9: 116-125.

Sambrook, J. F. E., Maniatis T. 1989, Molecular Cloning: a Laboratory Manual. Cold Spring Harbor press, NY.

Schiestl, R. H. and R. D. Gietz. 1989, Curr. Genet. 16: 339-346.

Schuller, C., J. L. Brewster, M. R. Alexander, M. C. Gustin, and H. Ruis. 1994, EMBO J. 13: 4382-4389.

Silhavy T J, B. M., Enquist L W. 1984. Experiments with gene fusion. In pp. 75-78. Cold Spring Harbor press, NY.

Stanhill, A., N. Schick, and D. Engelberg. 1999, Mol. Cell. Biol. 19: 7529-7538.

Tamura, K., T. Sudo, U. Senftleben, A. M. Dadak, R. Johnson, and M. Karin. 2000, Cell 102: 221-231.

Tanoue, T., M. Adachi, T. Moriguchi, and E. Nishida. 2000, Nat. Cell. Biol. 2: 110-116.

Tournier, C., P. Hess, D. D. Yang, J. Xu, T. K. Turner, A. Nimnual, D. Bar-Sagi, S. N. Jones, R. A. Flavell, and R. J. Davis. 2000, Science 288: 870-874.

Wang, Z., P. C. Harkins, R. J. Ulevitch, J. Han, M. H. Cobb, and E. J. Goldsmith. 1997, Proc. Natl. Acad. Sci. USA 94: 2327-2332.

Widmann, C., S. Gibson, M. B. Jarpe, and G. L. Johnson. 1999, Physiol. Rev. 79: 143-180.

Yang, D. D., D. Conze, A. J. Whitmarsh, T. Barrett, R. J. Davis, M. Rincon, and R. A. Flavell. 1998, Immunity 9: 575-585.

Zheng, C., J. Xiang, T. Hunter, and A. Lin. 1999, J. Biol. Chem. 274: 28966-28971.

1. A method of screening for a constitutively activated mutant of a desired eukaryotic MAPK pathway member of a MAPK (mitogen activated protein kinase) pathway, which method comprises the steps of:
a. providing a mutant yeast strain devoid of an upstream kinase, wherein said upstream kinase is capable of activating the MAPK pathway member of a yeast MAPK pathway that is equivalent or corresponding to said desired eukaryotic MAPK pathway member;
b. providing a DNA library of different mutants of a mutagenized gene coding for said desired MAPK pathway member;
c. introducing said library of (b) into said mutant yeast strain of (a) under conditions suitable for activation of said yeast MAPK pathway;
d. detecting an end-point indication of the activation of said yeast MAPK pathway, wherein said activation results from rescuing said yeast MAPK pathway by a constitutively activated mutant of the desired eukaryotic MAPK pathway member; and
e. optionally, isolating said constitutively activated mutant from selected rescued clones.

2. The method according to claim 1, wherein the activation of said constitutively activated mutant is independent of the presence of a kinase upstream thereto, which upstream kinase is capable of phosphorylating and activating said MAPK pathway member.

3. The method according to claim 2, wherein said yeast is selected from the group consisting of Saccharomyces cerevisiae and Schizosaccharomyces pombe.

4. The method according to claim 3, wherein the end-point indication is any one of ability of the cells to survive under high osmotic conditions, ability to mate, ability to invade agar, the ability to form pseudohyphae, the ability of the yeast to sporulate and any combinations thereof.

5. The method according to claim 4, wherein said desired eukaryotic MAPK pathway member is selected from the group consisting of mammalian, plant cells, avian cells, insect cells, fungi cells and yeast cell MAPKs pathway members.

6. The method according to claim 5, wherein said desired eukaryotic MAPK pathway member is any one of MAPKs and MAPKKs (MAPK Kinases).

7. The method according to claim 6, wherein said MAPK pathway member is a MAPK.

8. The method according to claim 7, wherein said MAPK pathway member is a MAPK selected from the group consisting of plant cells, insect cells, avian cells or mammalian ERK1, ERK2, ERK 5, ERK 7, JNK, p38 subfamilies, the yeast Fus3, Kss1, Mpk1 and Hog1.

9. The method according to claim 8, wherein said MAPK is any one of plant, insect, avian or mammalian ERK subfamily member, or the yeast Fus3, Kss1 and Mpk1, which method comprises the steps of:
a. providing the mutant yeast strain ste7Δ which is devoid of the upstream kinase MAPKK, Ste7, wherein said Ste7 is capable of activating the MAPK pathway member of a yeast MAPK pathway;
b. providing a DNA library of different mutants of a mutagenized gene coding for any one of said yeast, fungi, plant, avian, insect and mammalian MAPK,;
c. introducing said library of (b) into said ste7Δ yeast strain of (a) under suitable conditions for activation of said yeast MAPK pathway;
d. detecting an end-point indication for activation of the yeast MAPK pathway, wherein said activation results from rescuing said yeast MAPK pathway by a constitutively activated mutant of said MAPK; and
e. optionally, isolating said constitutively activated MAPK mutant from selected rescued clones.

10. The method according to claim 9, wherein said yeast MAPK pathway is the Ste7/Fus3 pathway and the end-point indication is the ability of rescued cells to mate.

11. The method according to claim 9, wherein said yeast MAPK pathway is the Ste7/Kss1 pathway and the end-point indication is the ability of rescued cells to invade agar and to form pseudohyphae.

12. The method according to claim 8, wherein said MAPK is any one of plant, insect, avian or mammalian JNK or p38 subfamilies member, and the yeast Hog1, which method comprises the steps of:
a. providing the mutant yeast strain pbs2, which is devoid of the upstream MAPKK, Pbs2, wherein said Pbs2 is capable of activating the MAPK pathway member of a yeast MAPK pathway;
b. providing a DNA library of different mutants of a mutagenized gene coding for one of said yeast, plant, insect, avian and mammalian MAPK;
c. introducing said library of mutants of (b) into said pbs2Δ yeast strain of (a) under suitable conditions for activation of said yeast MAPK pathway;
d. detecting an end-point indication for activation of said yeast MAPK pathway, wherein said activation results from rescuing said pathway by a constitutively activated mutant of said MAPK; and
e. optionally, isolating said constitutively activated MAPK mutant from said selected rescued clones.

13. The method according to claim 12, wherein said yeast MAPK pathway is the Pbs2/Hog1 and the end-point indication is the ability of rescued cells to survive under high osmotic conditions.

14. The method according to claim 13, wherein said MAPK is the yeast Hog1, which method comprises the steps of:
a. providing the mutant yeast strain pbs2Δ which is devoid of the upstream MAPKK, Pbs2, wherein said Pbs2 is capable of activating Hog1;
b. providing a DNA library of different mutants of a mutagenized HOG1 gene coding for the MAPK Hog1;
c. introducing said library of mutants of (b) into said pbs2Δ yeast strain of (a) under suitable conditions for activation of said yeast Pbs2/Hog1 pathway;
d. detecting an end-point indication for activation of the yeast Pbs2/Hog1 pathway, wherein said activation results from rescuing said pathway by a constitutively activated Hog1 mutant; and
e. optionally, isolating said constitutively activated Hog1 mutant from selected rescued clones.

15. The method according to claim 14, wherein said constitutively activated Hog1 mutant is capable of activating the authentic pathway downstream to Hog1 independently of Pbs2 and endogenous Hog1.

16. The method according to claim 15, wherein said mutant has at least one mutation selected from the group consisting of point mutation, missense, nonsense, insertion, deletions and rearrangement.

17. The method according to claim 16, wherein said mutant carries at least one mutation in the conserved L16 domain of the protein.

18. The method according to claim 17, wherein said mutation is between residues 314 to 332 of said L16 domain, which said residues are located within the Hog1 amino acid sequence as substantially denoted by SEQ ID NO. 1.

19. The method according to claim 18, wherein said mutant has at least one point mutation located at any position of A314, F318, W320, P322, W332 and any combinations thereof, within the Hog1 amino acid sequence as substantially denoted by SEQ ID NO. 1.

20. The method according to claim 19, wherein said mutant has at least one point mutation selected from the group consisting of A314T, F318L, P318S, W320R, F322L, W332R and any combinations thereof, which point mutations are located within the Hog1 amino acid sequence as substantially denoted by SEQ ID NO. 1.

21. The method according to claim 16, wherein said mutant has at least one point mutation located at any position of Y68, D170, N391 and any combinations thereof, within the Hog1 amino acid sequence as substantially denoted by SEQ ID NO. 1.

22. The method according to claim 21, wherein said mutant has at least one point mutation selected from the group consisting of Y68H, D170A, N391D and any combinations thereof, which point mutations are located within the Hog1 amino acid sequence as substantially denoted by SEQ ID NO. 1.

23. The method according to claim 6, wherein said MAPK pathway member is a MAPKK.

24. The method according to claim 23, wherein said MAPKK is selected from the group consisting of plant, insect, avian or mammalian MEK, JNKK, the yeast Ste7, Pbs2 and Byr1, which method comprises the steps of:
a. providing a mutant yeast strain MAPKKKΔ, devoid of MAPKKK which is the kinase upstream to MAPKK, wherein said MAPKKK is capable of activating the MAPKK of said yeast MAPK pathway;
b. providing a DNA library of different mutants of a mutagenized gene coding for one of said yeast, fungi, plant, insect, avian or mammalian MAPKKS;
c. introducing said library of mutants of (b) into said MAPKKKΔyeast strain of (a) under suitable conditions for activation of said yeast MAPK pathway;
d. detecting an end-point indication for activation of the yeast MAPK pathway, wherein said activation results from rescuing said pathway by a constitutively activated mutant of the MAPKK; and
e. optionally, isolating said constitutively activated MAPKK mutant from selected rescued clones.

25. A constitutively activated mutant according to a MAPK pathway member of a MAPK pathway, capable of activating said MAPK pathway independently of a kinase upstream thereto.

26. The activated mutant according to claim 25, wherein said mutant has at least one mutation selected from the group consisting of point mutation, missense nonsense, insertion, deletion and rearrangement.

27. The activated mutant according to claim 26, wherein said MAPK pathway member is a eukaryotic MAPK pathway member selected from the group consisting of mammalian, plant cells, insect cells, avian cells, fungi cells and yeast cells MAPK pathway members.

28. The activated mutant according to claim 27, wherein said MAPK pathway member is any one of MAPK and MAPKK.

29. The activated mutant according to claim 28, wherein said MAPK pathway member is a MAPK.

30. The activated mutant according to claim 29, wherein said MAPK is selected from the group consisting of mammalian ERK1, ERK2, ERK5, ERK7, JNK, p38 subfamilies member, the yeast Fus3, Kss1, Mpk1 and Hog1.

31. The activated MAPK mutant according to claim 30, wherein said Hog1 mutant is capable of activating the authentic pathway downstream to Hog1 independently of Pbs2 and endogenous Hog1.

32. The activated MAPK mutant according to any one of claims 30 and 31, wherein said mutant has at least one mutation occurring in the conserved L16 domain of the Hog1 protein or of any said MAPK.

33. The activated MAPK mutant according to claim 32, wherein said mutation is between residues 314 to 332 of said L16 domain or in the respective L16 domain of any of said MAPK, which said residues are located within the Hog1 amino acid sequence as substantially denoted by SEQ ID NO. 1.

34. The activated MAPK mutant according to claim 33, wherein said mutant has at least one point mutation in Hog- or a respective mutation in any equivalent MAPK, which mutation is located at any position of A314, F318, W320, F322, W332 and any combinations thereof, which said mutations are located within the Hog1 amino acid sequence as substantially denoted by SEQ ID NO. 1.

35. The activated MAPK mutant according to claim 34, wherein said Hog1 mutant carries at least one point mutation selected from the group consisting of A314T, F318L, F318S, W320R, F322L, W332R and any combination thereof, which said mutations are located within the Hog1 amino acid sequence as substantially denoted by SEQ ID NO. 1.

36. The activated MAPK mutant according to claim 34, wherein said respective mutation in any MAPK is located at any position of A342, F346 of the human ERK1, A325, F329 of the human ERK2, A323, F327 of the rat ERK2, W352 of the human JNK1 and F327, W337, and A320 of the human or the rat p38 and any combination thereof.

37. The activated MAPK mutant according to claim 36, wherein said respective mutation in any MAPK is selected from the group consisting of A342T, F346L and F346S of the human ERK1, A325T, F329L and F329S of the human ERK2, A323T, F327S and F327L of the rat ERK2, W352R of the human JNK1 and F327L, F327S, W337R, and A320T of the human or the rat p38 and any combination thereof.

38. The activated MAPK mutant according to claim 37, wherein said A320T, W337R, F327S and F327L are point mutations in the human p38.

39. The activating mutant according to any of claims 30 and 31, wherein said mutant carries at least one point mutation in Hog1 or a respective mutation in any MAPK, which mutation is located at any position of Y68, D170 and N391.

40. The activating mutant according to claim 39, wherein said Hog1 mutant has at least one point mutation selected from the group consisting of Y68H, D170A, N391D and any combinations thereof, which said mutations are located within the Hog1 amino acid sequence substantially as denoted by SEQ ID NO. 1.

41. The activated MAPK mutant according to claim 39, wherein said respective mutation in any of said MAPK is located at any position of D192 of the human ERK1, D175 of the human ERK2, D173 of the rat ERK2, Y71 of the human JNK1, Y68 and D176 of the human or rat p38.

42. The activated MAPK mutant according to claim 41, wherein said respective mutation in any of said MAPK is selected from the group consisting of D192A of the human ERK1, D175A of the human ERK2, D173A of the rat ERK2, Y71H of the human JNK1, Y68H and D 176A of the human or rat p38.

43. An activated MAPK or MAPKK obtained by the method of any one of claims 1 to 24.

44. A method of producing a desired constitutively activated mutant of an eukaryotic MAPK pathway member, which method comprises the steps of:
a. providing an activated MAPK pathway member according to claim 43;
b. subjecting the activated mutant provided in step (a) to nucleic acid sequence analysis;
c. aligning the sequence obtained in step (b) with the nucleic acid sequence of said desired MAPK pathway member; and
d. introducing to said desired MAPK pathway member a mutation equivalent to the mutation in said activated MAPK provided in (a).

45. A constitutively activated mutant of an eukaryotic MAPK pathway member produced by the method of claim 44.

46. An expression vector comprising a nucleic acid sequence coding for a constitutively activated mutant of a MAPK pathway member operably linked to a promoter, terminator, any additional control, promoting and/or regulatory elements and optionally a selectable marker and/or a tag sequence.

47. The expression vector according to claim 46, wherein said activated mutant of a MAPK pathway member is as defined by any of claims 25 to 43 and 45.

48. The expression vector according to claim 47, wherein said promoter is any one of a constitutive and an inducible promoter.

49. A host cell transformed with an expression vector as defined in any one of claims 46 to 48.

50. The host cell according to claim 49, which is any one of prokaryotic and eukaryotic cell.

51. The host cell according to claim 50, which is any one of bacterial cell, yeast cell, an insect cell, avian cell a plant cell or a mammalian cell.

52. A non-human transgenic organism carrying a DNA sequence or an expression vector comprising the same, said DNA sequence coding for a constitutively activated mutant of a MAPK pathway member.

53. The transgenic organism according to claim 52, wherein said activated mutant of a MAPK pathway member is as defined in any of claims 25 to 43 and 45.

54. The transgenic organism according to claim 53, wherein the expression vector is as defined in any of claims 46 to 48.

55. A recombinant protein comprising a constitutively activated mutant of a MAPK pathway member capable of activating said MAPK pathway independently of the presence of a kinase upstream thereto.

56. The recombinant protein according to claim 55, wherein said MAPK pathway member is any one of MAPK and MAPKK.

57. The recombinant protein according to claim 56, wherein said mutant of a MAPK pathway is as defined in any one of claims 25 to 43 and 45.

58. A method of screening for a substance which is an inhibitor of a MAPK pathway, which method comprises the steps of:
a. providing a mixture comprising a constitutively activated mutant of a MAPK pathway member or any functional fragments thereof;
b. contacting said mixture with a test substance under conditions suitable for activation of said MAPK pathway;
c. determining the effect of the test substance on an end-point indication, wherein said effect is indicative of inhibition of said MAPK pathway by the test substance.

59. The method according to claim 58, wherein said constitutively activated mutant of a MAPK pathway member is an activated mutant of any one of MAPK and MAPKK.

60. The method according to claim 59, wherein said activated mutant of MAPK or MAPKK is as defined in any one of claims 25 to 43 and 45.

61. The method according to claim 60, wherein said mixture is a cell mixture or a cell-free mixture.

62. The method according to claim 61, wherein said mixture comprises:
a. a constitutively activated mutant of any one of MAPK, MAPKK and any functional fragments thereof;
b. an interactor molecule which can interact with said activated mutant, wherein the interaction of said interactor with said activated molecule indicates activation of said MAPK pathway by the activated mutant; and
c. optionally further solutions, buffers and compounds which provide suitable conditions for activation of said MAPK pathway and for the detection of an end-point indication for the interaction of said activated mutant with said interactor molecule;
whereby interaction of said interactor molecule with said activated mutant is detected by an end-point indication.

63. The method according to claim 62, wherein interaction of the activated mutant with the interactor molecule in the presence of the test substance is detected by the end-point indication, whereby inhibition of said end-point indicates inhibition of the MAPK pathway by said test substance.

64. The method according to claim 63, wherein the reaction mixture is a cell-free mixture.

65. The method according to claim 64, wherein said activated mutant or any functional fragment thereof, is provided as a purified recombinant protein as defined in any one of claims 55 to 57, as a fusion protein or as a cell lysate of a transformed host cell as defined in any one of claims 49 to 51, expressing said activated mutant or any functional fragments thereof.

66. The method according to claim 65, wherein said interactor molecule comprises a downstream substrate of said MAPK pathway member.

67. The method according to claim 66, wherein the step of detecting the interaction of said interactor substrate molecule with said activated mutant, includes detecting an enzymatic activity of said activated mutant.

68. A method of screening for a substance which is an inhibitor of a MAPK pathway according to any of claims 64 to 67, which method comprises the steps of:
a. providing a cell-free mixture comprising a constitutively activated mutant of a MAPK pathway member and a fusion protein of a respective downstream substrate of said MAPK pathway member;
b. contacting said mixture with a test substance under conditions suitable for an in vitro kinase assay;
c. determining the effect of the test substance on phosphorylation of said substrate by the activated mutant, as an end-point indication, whereby inhibition of the phosphorylation indicates inhibition of said MAPK pathway by the test substance.

69. The method according to claim 63, wherein said reaction mixture is a cell mixture.

70. The method of screening according to claim 69, which method comprises the steps of:
a. providing a cell mixture comprising a constitutively activated mutant of a MAPK pathway member and an interactor molecule as defined in claim 62;
b. contacting said cell mixture with a test substance;
c. detecting an interaction of the activated mutant with the interactor molecule in the presence of the test substance by searching for an end-point indication, whereby inhibition of said end-point indicates inhibition of the MAPK pathway by said test substance.

71. The method according to claim 70, wherein said cell mixture is a recombinant cell transformed by:
a. an expression vector as defined in any one of claims 46 to 48, comprising a nucleic acid sequence coding for the activated mutant of a MAPK pathway member as defined in any one of claims 25 to 43 and 45; and
b. a construct comprising an interactor molecule, wherein said interactor molecule comprises:
(i) a transcriptional regulatory sequence;
(ii) operably linked reporter gene, and
c. optionally, further endogenously or exogenously expressible interactor molecules essential for activation of said pathway.

72. The method according to claim 71, wherein interaction of said mutant of MAPK pathway member with downstream signaling molecules results in mediation of transcription of said reporter gene, wherein the transcription is driven by said regulatory sequence.

73. The method according to claim 72, wherein the end-point indication is the expression of said reporter gene, which leads to a visually detectable signal.

74. The method according to claim 73, wherein decrease of said detectable signal in the presence of the test substance indicates inhibition of the MAPK pathway by said test substance.

75. The method according to claim 70, which method comprises the steps of:
a. providing recombinant cell mixture comprising a constitutively activated mutant of a MAPK pathway member and at least one downstream interactor molecule essential for transducing a signal through said pathway;
b. contacting said recombinant cell mixture with a test substance; and
c. determining the effect of said test substance on the interaction of the activated mutant with the interactor molecule by searching the end-point indication, wherein said effect is indicative of inhibition of the MAPK pathway by said test substance.

76. The method according to claim 75, wherein said end-point indication is a cell phenotype caused by activation of said MAPK pathway.

77. The method according to claim 76, wherein said activated mutant is optionally expressed under an inducible promoter.

78. The method according to claim 77, wherein said recombinant cell mixture is a yeast cell culture.

79. The method according to claim 78, wherein the end-point indication is any one of ability of the cells to proliferate, ability to survive under high osmotic conditions, ability to mate, ability to invade agar and to form pseudohyphae, the ability of the yeast to sporulate and any combinations thereof.

80. The method according to claim 77, wherein said recombinant cell is any one of plant cell, insect cell, avian cells and a mammalian cell.

81. The method according to claim 80, wherein said end-point indication is any one of ability to proliferate, ability to induce apoptosis, ability to induce oncogenic phenotype and ability to induce differentiation.

82. The method according to any one of claims 58 to 81, wherein said test substance is selected from the group consisting of protein based, carbohydrates based, lipid based, nucleic acid based, natural organic based, synthetically derived organic based, antibody based, inorganic based and peptidomimetics based substances.

83. The method according to claim 82, wherein said protein based substance is a product of any one of positional scanning of peptide libraries, libraries of cyclic peptidomimetics, peptide combinatorial libraries and phage display random or dedicated libraries.

84. The method according to any of claims 82 and 83, wherein said test substance is an inhibitor of said MAPK pathway.

85. A method of preparing a therapeutic composition for the inhibition of a MAPK pathway in a mammalian subject in need of such treatment, which method comprises the steps of:
a. identifying an inhibitor of a MAPK pathway; and
b. admixing said inhibitor substance with at least one of a pharmaceutically acceptable carrier, diluent, excipient and/or additive.

86. The method according to claim 85, wherein said inhibitor substance is identified by the screening method defined in any one of claims 68 to 83.

87. The method according to claim 86, wherein the therapeutic composition is for the treatment of a pathological disorder selected from the group consisting of neoplasia, cancer, inflammation, degenerative diseases and immunological disorders.


SEQUENCE LISTING (22 sequences)

SEQ ID NO: 1 - protein, 435 aa, Yeast
MTTNEEFIRTQIFGTVFEITNRYNDLNPVGMGAFGLVCSATDTLTSQPVAIKKIMKPFSTAVLAKRTYRELKLLKHLRHENLICLQDIFLSPLEDIYFVTELQGTDLHRLLQTRPLEKQFVQYFLYQILRGLKYVHSAGVIHRDLKPSNILINENCDLKICDFGLARIQDPQMTGYVSTRYYRAPEIMLTWQKYDVEVDIWSAGCIFAEMIEGKPLFPGKDHVHQFSIITDLLGSPPKDVINTICSENTLKFVTSLPHRDPIPFSERFKTVEPDAVDLLEKMLVFDPKKRITAADALAHPYSAPYHDPTDEPVADAKFDWHFNDADLPVDTWRVMMYSEILDFHKIGGSDGQIDISATFDDQVAAATAAAAQAQAQAQAQVQLNMAAHSHNGAGTTGNDHSDIAGGNKVSDHVAANDTITDYGNQAIQYANEFQQ

SEQ ID NO: 2 - DNA, 37 bp, Artificial Sequence
  Description of Artificial SequencePRIMER
tccactgcagtgctggccaaaaggacacatctggaac

SEQ ID NO: 3 - DNA, 22 bp, Artificial Sequence
  Description of Artificial SequencePRIMER
ccaaatgtcgacctcgacgtca

SEQ ID NO: 4 - DNA, 31 bp, Artificial Sequence
  Description of Artificial SequencePRIMER
cggtctagcaagaattcaagcccctcaaatg

SEQ ID NO: 5 - DNA, 34 bp, Artificial Sequence
  Description of Artificial SequencePRIMER
ccaacggatgaaccagtaaccgatgccaagttcg

SEQ ID NO: 6 - DNA, 34 bp, Artificial Sequence
  Description of Artificial SequencePRIMER
cgaacttggcatgccttactggttcatccgttgg

SEQ ID NO: 7 - DNA, 33 bp, Artificial Sequence
  Description of Artificial SequencePRIMER
gaaccagtagccgatgccaagctcgattggcac

SEQ ID NO: 8 - DNA, 33 bp, Artificial Sequence
  Description of Artificial SequencePRIMER
gtgccaatcgagcttggcatcggctactggttc

SEQ ID NO: 9 - DNA, 33 bp, Artificial Sequence
  Description of Artificial SequencePRIMER
gaaccagtagccgatgccaagtccgattggcac

SEQ ID NO: 10 - DNA, 33 bp, Artificial Sequence
  Description of Artificial SequencePRIMER
gtgccaatcggacttggcatcggctactggttc

SEQ ID NO: 11 - DNA, 33 bp, Artificial Sequence
  Description of Artificial SequencePRIMER
cgatgccaagttcgatcggcactttaatgacgc

SEQ ID NO: 12 - DNA, 33 bp, Artificial Sequence
  Description of Artificial SequencePRIMER
gcgtcattaaagtgccgatcgaacttggcatcg

SEQ ID NO: 13 - DNA, 34 bp, Artificial Sequence
  Description of Artificial SequencePRIMER
gccaagttcgattggcaccttaatgacgctgatc

SEQ ID NO: 14 - DNA, 34 bp, Artificial Sequence
  Description of Artificial SequencePRIMER
gatcagcgtcattaaggtgccaatcgaacttggc

SEQ ID NO: 15 - DNA, 36 bp, Artificial Sequence
  Description of Artificial SequencePRIMER
gatctgcctgtcgatacccggcgtgttatgatgtac

SEQ ID NO: 16 - DNA, 36 bp, Artificial Sequence
  Description of Artificial SequencePRIMER
gtacatcataacacgccgggtatcgacaggcagatc

SEQ ID NO: 17 - DNA, 46 bp, Artificial Sequence
  Description of Artificial SequencePRIMER
caagaccctcaaatggcaggctttgtttccactagatactacaggg

SEQ ID NO: 18 - DNA, 46 bp, Artificial Sequence
  Description of Artificial SequencePRIMER
ccctgtagtatctagtggaaacaaagcctgccatttgagggtcttg

SEQ ID NO: 19 - DNA, 35 bp, Artificial Sequence
  Description of Artificial SequencePRIMER
gtctagcaagaattcaagcccctcaaatgacaggc

SEQ ID NO: 20 - DNA, 35 bp, Artificial Sequence
  Description of Artificial SequencePRIMER
gcctgtcatttgaggggcttgaattcttgctagac

SEQ ID NO: 21 - DNA, 37 bp, Artificial Sequence
  Description of Artificial SequencePRIMER
tccactgcagtgctggccaaaaggacacatcgtgaac

SEQ ID NO: 22 - DNA, 33 bp, Artificial Sequence
  Description of Artificial SequencePRIMER
ctagccggatccttactgttggaactcattagc